(19) United States
(12) Patent Application Publication
(10) Pub. No.: US 2021/0024909 A1
(43) Pub. Date: Jan. 28, 2021

(54) CHIMERIC AMYLASES COMPRISING AN HETEROLOGOUS STARCH BINDING DOMAIN

(71) Applicant: Lallemand Hungary Liquidity Management LLC, Budapest (HU)

(72) Inventors: Ryan Skinner, Bethel, VT (US); Charles F. Rice, Plainfield, NH (US); Aaron Argyros, Lebanon, NH (US)

(21) Appl. No.: 17/041,392

(22) PCT Filed: Mar. 25, 2019
(86) PCT No.: PCT/IB2019/052410

§ 371 Date: Sep. 24, 2020

Related U.S. Application Data
(60) Provisional application No. 62/648,243, filed on Mar. 26, 2018.

(51) Int. Cl. C12N 9/28; C12N 9/34; C12P 7/06

(52) U.S. Cl. CPC C12N 9/2417; C12P 7/06; C12N 9/2428

(57) ABSTRACT

The present disclosure relates to chimeric polypeptides for improving the hydrolysis of starch. The chimeric polypeptides has an alpha-amylase linked to a starch binding domain. The chimeric polypeptides can be provided in a purified form and/or can be expressed from 5 a recombinant host cell. The present disclosure also provides a population of recombinant host cells expressing the chimeric polypeptides.

### TECHNOLOGICAL FIELD

The present disclosure relates to enzymes, such as amylases, fused with a starch binding domain that can be used for improving the hydrolysis of starch.

### BACKGROUND

Saccharomyces cerevisiae is the primary biocatalyst used in the commercial production of fuel ethanol. This organism is proficient in fermenting glucose to ethanol, often to concentrations greater than 20% w/v. However, S. cerevisiae lacks the ability to hydrolyze polysaccharides and therefore requires the exogenous addition of purified enzymes to convert complex sugars to glucose. For example, in the United States, the primary source of fuel ethanol is corn starch, which, regardless of the mashing process, requires the exogenous addition of both alpha-amylases and glucoamylases. The cost of the purified enzymes range from $0.02-0.04 per gallon, which, at 14 billion gallons of ethanol produced each year, represents a substantial cost savings opportunity for producers if they could reduce their enzyme dose.

Glucoamylases (EC 3.2.1.3) are exo-acting enzymes which take starch to glucose, while alpha-amylases (EC 3.2.1.1) are endo-acting, taking starch to maltose and maltodextrins (Ghang et al. 2007), Saccharomyces cerevisiae strains engineered to secrete heterologous glucoamylase and alpha-amylase enzymes simultaneously are able to sufficiently break down starch to glucose, while simultaneously fermenting glucose to ethanol. This balance between hydrolysis and fermentation keeps the presence of reducing sugars low, reducing osmotic stress on the cell (Birol et al. 1998). In addition to increasing process efficiency, co-expression of these distinct but complimentary enzymes is able to reduce the need for addition of expensive amylase mixtures, as well as reduce the need for the energy-intensive step of heating the raw material to temperatures approaching 180° C. (Shigechi et al. 2004).

It would be desirable to improve the activity of alpha-amylases so as to reduce the amount of exogenous enzymes to be added in purified form for an ethanol production process from starch. It would further be desirable to provide alpha-amylase enzymes in a recombinant yeast cell host.

### BRIEF SUMMARY

The present disclosure relates to alpha-amylases with enhanced activity for the hydrolysis of starch, including raw starch. This is achieved by fusing a starch-binding domain to an alpha-amylase to provide a chimeric protein intended to be expressed in a recombinant yeast host cell.

In a first aspect, the present disclosure provides a chimeric polypeptide having alpha-amylase activity. The chimeric polypeptide comprises (i) a polypeptide having alpha-amylase activity (AA); associated with (ii) a starch binding domain (SBD) moiety. In some embodiments, the chimeric polypeptide is a chimeric polypeptide of formula (I):

(NH₂)SS-AA-L-SBD(COOH) (I)

wherein SS is an optional signal sequence (which is cleaved upon the secretion of the chimeric polypeptide outside the recombinant yeast host cell); AA is an alpha-amylase polypeptide moiety, a variant thereof, or a fragment thereof and has alpha-amylase activity; L is an optional amino acid linker; SBD is a starch binding domain moiety, a variant thereof, or a fragment thereof; (NH₂) indicates the amino terminus of the chimeric polypeptide; (COOH) indicates the carboxyl terminus of the chimeric polypeptide; and “-” is an amide linkage. In another embodiment, the chimeric polypeptide is provided having alpha-amylase activity, wherein the chimeric polypeptide is a chimeric polypeptide of formula (II):

(NH₂)SS-SBD-L-AA(COOH) (II)

wherein SS is an optional signal sequence (which is cleaved upon the secretion of the chimeric polypeptide outside the recombinant yeast host cell); AA is an alpha-amylase polypeptide moiety, a variant thereof, or a fragment thereof and has alpha-amylase activity; L is an optional amino acid linker; SBD is a starch binding domain moiety, a variant thereof, or a fragment thereof; (NH₂) indicates the amino terminus of the chimeric polypeptide; (COOH) indicates the carboxyl terminus of the chimeric polypeptide; and “-” is an amide linkage. In an embodiment, the chimeric polypeptide comprises the SS and is a chimeric polypeptide of formula (IA):

(NH₂)SS-AA-SBD(COOH) (IA).

In an embodiment, the chimeric polypeptide comprises the SS and is a chimeric polypeptide of formula (IIA):

(NH₂)SS-SBD-AA(COOH) (IIA).

In an embodiment, the SS has the amino acid sequence of SEQ ID NO: 6, is a variant of the amino acid sequence of SEQ ID NO: 6, or is a fragment of the amino acid sequence of SEQ ID NO: 6. In an embodiment, the SS has the amino acid sequence of SEQ ID NO: 13, is a variant of the amino acid sequence of SEQ ID NO: 13, or is a fragment of the amino acid sequence of SEQ ID NO: 13. In an embodiment, the SS has the amino acid sequence of SEQ ID NO: 14, is a variant of the amino acid sequence of SEQ ID NO: 14, or is a fragment of the amino acid sequence of SEQ ID NO: 14. In an embodiment, the SS has the amino acid sequence of SEQ ID NO: 15, is a variant of the amino acid sequence of SEQ ID NO: 15, or is a fragment of the amino acid sequence of SEQ ID NO: 15. In still another embodiment, the chimeric polypeptide lacks the SS. In such embodiment, the chimeric polypeptide comprising L and is a chimeric polypeptide of formula (IB):

(NH₂)AA-L-SBD(COOH) (IB).

In another embodiment, the chimeric polypeptide comprising L and is a chimeric polypeptide of formula (IIB):

(NH₂)SBD-L-AA(COOH) (IIB).

In an embodiment of the chimeric polypeptide, the L comprises one or more glycine residues. In an embodiment of the chimeric polypeptide, the L comprises one or more serine residues. In an embodiment, the L has the amino acid sequence of SEQ ID NO: 16, 17, 18, 19, 20, 21, 22, 23, or 24; is a variant of the amino acid sequence of SEQ ID NO: 16, 17, 18, 19, 20, 21, 22, 23, or 24; or is a fragment of the amino acid sequence of SEQ ID NO: 16, 17, 18, 19, 20, 21, 22, 23, or 24. In an embodiment of the chimeric polypeptide, the polypeptide having AA activity is from a Bacillus sp. alpha-amylase, a variant thereof, or a fragment thereof. In an embodiment of the chimeric polypeptide, the polypeptide having AA is from a Bacillus amyloliquefaciens alpha-amylase, a variant thereof, or a fragment thereof. In an embodiment of the chimeric polypeptide, the polypeptide having AA is from a Bacillus amyloliquefaciens amyE alpha-amylase, a variant thereof, or a fragment thereof. In an embodiment of the chimeric polypeptide, the polypeptide having AA has the amino acid sequence of SEQ ID NO: 3, is a variant of the amino acid sequence of SEQ ID NO: 3, or is a fragment of the amino acid sequence of SEQ ID NO: 3. In an embodiment of the chimeric polypeptide, the polypeptide having AA has the amino acid sequence of SEQ ID NO: 12, is a variant of the amino acid sequence of SEQ ID NO: 12, or is a fragment of the amino acid sequence of SEQ ID NO: 12. In an embodiment of the chimeric polypeptide, the SBD moiety has high binding affinity to raw starch. In an embodiment of the chimeric polypeptide, the SBD moiety enhances the activity of the polypeptide having AA on raw starch when compared to the activity of a polypeptide having alpha-amylase activity and lacking the SBD moiety (including variants and fragments thereof). In an embodiment of the chimeric polypeptide, the SBD moiety is derived from a glucoamylase enzyme, a variant thereof, or a fragment thereof. In an embodiment of the chimeric polypeptide, the SBD moiety is derived from an Aspergillus sp. glucoamylase, a variant thereof, or a fragment thereof. In an embodiment of the chimeric polypeptide, the SBD moiety is derived from an Aspergillus niger glucoamylase G1, a variant thereof, or a fragment thereof. In an embodiment of the chimeric polypeptide, the SBD moiety has the amino acid sequence of SEQ ID NO: 7, is a variant of the amino acid sequence of SEQ ID NO: 7, or is a fragment of the amino acid sequence of SEQ ID NO: 7. In an embodiment of the chimeric polypeptide, the chimeric polypeptide can comprise the amino acid sequence of SEQ ID NO: 5, be a variant of the amino acid sequence of SEQ ID NO: 5, or be a fragment of the amino acid sequence of SEQ ID NO: 5. In a specific embodiment, the chimeric polypeptide comprises the polypeptide having AA from Bacillus amyloliquefaciens amyE alpha-amylase (and can have, for example, the amino acid sequence of SEQ ID NO: 3 or 12, be a variant thereof or be a fragment thereof) and the SBD moiety can be derived from Aspergillus niger glucoamylase G1 (and can have, for example, the amino acid sequence of SEQ ID NO: 7, be a variant thereof or a fragment thereof). In an embodiment of the chimeric polypeptide, the chimeric polypeptide can comprise the amino acid sequence of SEQ ID NO: 8, be a variant of the amino acid sequence of SEQ ID NO: 8, or be a fragment of the amino acid sequence of SEQ ID NO: 8. In an embodiment of the chimeric polypeptide is provided in a purified form or expressed from an heterologous nucleic acid molecule encoding the chimeric polypeptide in a recombinant host (e.g., yeast) cell. In an embodiment of the chimeric polypeptide, the recombinant host cell is from the genus Saccharomyces sp. and can be, in some additional embodiments, from the species Saccharomyces cerevisiae.

In a second aspect, there is provided an isolated nucleic acid molecule encoding the chimeric polypeptide. In an embodiment, the isolated nucleic acid molecule comprises the nucleotide sequence of SEQ ID NO: 1. In an embodiment, the isolated nucleic acid molecule comprises the nucleotide sequence of SEQ ID NO: 4.

In a third aspect, there is provided a recombinant (e.g., yeast) host cell having an heterologous nucleic acid molecule encoding (and optionally expressing) the chimeric polypeptide described herein. In an embodiment, the recombinant host cell is from the genus Saccharomyces sp. and can be, in some additional embodiments, from the species Saccharomyces cerevisiae.

In a fourth aspect, the present disclosure provides a purified, isolated and/or recombinant chimeric polypeptide obtained from a recombinant host cell described herein.

In a fifth aspect, the present disclosure provides a composition comprising the recombinant host cell described herein or the purified, isolated and/or recombinant chimeric polypeptide described herein and at least one of a glucoamylase or starch.

In a sixth aspect, the present disclosure provides a yeast product made from the recombinant yeast host cell described herein, comprising the purified, isolated and/or recombinant chimeric polypeptide described herein or the composition described herein. In an embodiment, the yeast product is an inactivated yeast product, such as, for example, a yeast extract.

In a seventh aspect, the present disclosure provides a process for hydrolyzing starch. The process comprises contacting the chimeric polypeptide, the recombinant host cell, the purified, isolated and/or recombinant chimeric polypeptide, the combination or the yeast product described herein with a medium comprising starch. In an embodiment of the process, the medium comprises raw starch. In an embodiment of the process, the medium is derived from corn. In an embodiment, the process comprises adding the recombinant host cell, the purified, isolated and/or recombinant polypeptide, the composition or the yeast product to a liquefaction medium. In an embodiment, the process comprises maintaining the liquefaction medium at a temperature of between about 25° C. and 60° C. during a period of time to obtain a liquefied medium. In a further embodiment, the process can be used for making a fermentation product from the liquefied medium. In such embodiment of the process, the process can further comprise fermenting the liquefied medium with a fermenting yeast cell to obtain the fermented product. In an embodiment of the process, the fermentation product is ethanol.

### BRIEF DESCRIPTION OF THE DRAWING

Having thus generally described the nature of the invention, reference will now be made to the accompanying drawing, showing by way of illustration, a preferred embodiment thereof, and in which:

FIG. 1 compares the total secreted amylase activity on corn flour (2%) from two strains: a first Saccharomyces cerevisiae strain (M9900) expressing two copies of Bacillus amyloliquefaciens amyE alpha-amylase (SE85); and a second Saccharomyces cerevisiae strain (M15747) expressing two copies of chimeric alpha-amylase (MP1032) comprising SE85 and both the linker and SBD regions from Aspergillus niger G1 glucoamylase.

### DETAILED DESCRIPTION

The present disclosure relates to polypeptides having enhanced alpha-amylase activity for the starch saccharification process (for example for improving the hydrolysis of starch, including the hydrolysis of starch, including raw starch). In particular, the present disclosure relates to chimeric polypeptides having alpha-amylase activity comprising a moiety having alpha-amylase activity fused with a starch binding domain (SBD) moiety.

When the chimeric polypeptides having alpha-amylase activity are used in combination with or expressed from heterologous nucleic acid molecules in one or more recombinant host cell capable of fermenting glucose to a fermentation product, such as ethanol (such as, for example, in a recombinant yeast host cell), in combination with a glucoamylase, it allows for the break-down of starch to glucose, while simultaneously fermenting glucose to ethanol. Chimeric polypeptides having alpha-amylase activity can also be used in the absence of a glucoamylase to liquefy a medium comprising starch.

The chimeric polypeptide of the present disclosure is heterologous with respect to the recombinant host cell that can be used to express it. The chimeric polypeptide is encoded by an heterologous nucleic acid molecule and can be expressed in a recombinant host cell including the heterologous nucleic acid molecule. The term “heterologous” when used in reference to a nucleic acid molecule (such as a promoter or a coding sequence) refers to a nucleic acid molecule that is not natively found in the recombinant host cell. “Heterologous” also includes a native coding region, or portion thereof, that is introduced into the source organism in a form that is different from the corresponding native gene, e.g., not in its natural location in the organism's genome. The heterologous nucleic acid molecule is purposively introduced into the recombinant host cell. The term “heterologous” as used herein also refers to an element (nucleic acid or protein) that is derived from a source other than the endogenous source. Thus, for example, an heterologous element could be derived from a different strain of host cell, or from an organism of a different taxonomic group (e.g., different kingdom, phylum, class, order, family genus, or species, or any subgroup within one of these classifications). The term “heterologous” is also used synonymously herein with the term “exogenous”.

In some embodiments, the chimeric polypeptide can be used in combination with an amylolytic enzyme. The “amylolytic enzyme”, an enzyme involved in amylase digestion, metabolism and/or hydrolysis. The amylolytic enzyme can be an amylase. The term “amylase” refers to an enzyme that breaks starch down into sugar. All amylases are glycoside hydrolases and act on α-1,4-glycosidic bonds. Some amylases, such as γ-amylase (glucoamylase), also act on α-1,6-glycosidic bonds. Amylase enzymes include α-amylase (EC 3.2.1.1), β-amylase (EC 3.2.1.2), and γ-amylase (EC 3.2.1.3). The α-amylases are calcium metalloenzymes, unable to function in the absence of calcium. By acting at random locations along the starch chain, α-amylase breaks down long-chain carbohydrates, ultimately yielding maltotriose and maltose from amylose, or maltose, glucose and “limit dextrin” from amylopectin. Because it can act anywhere on the substrate, α-amylase tends to be faster-acting than 3-amylase. Another form of amylase, β-amylase is also synthesized by bacteria, fungi, and plants. Working from the non-reducing end, β-amylase catalyzes the hydrolysis of the second α-1,4 glycosidic bond, cleaving off two glucose units (maltose) at a time. Another amylolytic enzyme is α-glucosidase that acts on maltose and other short malto-oligosaccharides produced by α-, β-, and γ-amylases, converting them to glucose.

Another amylolytic enzyme is pullulanase. Pullulanase is a specific kind of glucanase, an amylolytic exoenzyme, that degrades pullulan. Pullulan is regarded as a chain of maltotriose units linked by alpha-1,6-glycosidic bonds. Pullulanase (EC 3.2.1.41) is also known as pullulan-6-glucanohydrolase (debranching enzyme). Another amylolytic enzyme, isopullulanase, hydrolyses pullulan to isopanose (6-alpha-maltosylglucose). Isopullulanase (EC 3.2.1.57) is also known as pullulan 4-glucanohydrolase. An “amylase” can be any enzyme involved in amylase digestion, metabolism and/or hydrolysis, including α-amylase, β-amylase, glucoamylase, pullulanase, isopullulanase, and alpha-glucosidase.

### Chimeric Polypeptides

Chimeric polypeptides (also referred to a fusion proteins) are created through the joining of two or more polypeptides of different sources or different types of polypeptides, or are expressed from chimeric heterologous nucleic acid molecules created through the joining of two or more genes that encode different polypeptides or polypeptides of different sources. The chimeric polypeptides of the present disclosure have alpha-amylase activity and comprises joining a polypeptide moiety having alpha-amylase activity with a starch binding domain moiety having affinity for a starch molecule, such that the starch binding domain enhances the alpha-amylase activity of the chimeric polypeptide (when compared to the alpha-amylase activity of the alpha-amylase moiety in the absence of the starch-binding domain).

The chimeric polypeptides described herein are intended to be produced in a recombinant host cell and/or secreted by the recombinant host cell. Each of the components of the chimeric polypeptides comprise a stretch of consecutive amino acid residues, and the components are linked by amino bonds. Each of the components of the chimeric polypeptides may also comprise one or more polypeptides, and the components are linked by amino bonds. The chimeric polypeptides of the present disclosure comprise at least two moiety: a first one exhibiting alpha-amylase activity and a second one exhibiting starch binding activity (e.g., a starch binding domain). Chimeric polypeptides having the alpha-amylase activity can be used in a process to improve saccharification and/or the production of a fermentation product, such as ethanol, from starch (including raw starch).

In an embodiment, a chimeric polypeptide has formula (I):

(NH₂)SS-AA-L-SBD(COOH) (I)
wherein SS is an optional signal sequence (which is cleaved and removed from the chimeric polypeptide upon the secretion of the chimeric polypeptide by the recombinant host cell);
   AA is an alpha-amylase polypeptide, a variant thereof, or a fragment thereof and has alpha-amylase activity;
   L is an optional amino acid linker;
   SBD is a starch binding domain, a variant thereof, or a fragment thereof;
   (NH₂) indicates the amino terminus of the chimeric protein;
   (COOH) indicates the carboxyl terminus of the chimeric protein; and
   “-” is an amide linkage.

In formula (I), the carboxy terminus of the optional SS is (directly or indirectly) associated with the amino terminus of AA. The carboxy terminus of AA is (directly or indirectly) associated with the amino terminus of optional L. The carboxy terminus of optional L is (directly or indirectly) associated with the amino terminus of SBD. In one embodiment of the chimeric protein of formula (I), the carboxy terminus of the AA is directly associated with the amino terminus of the SBD.

In an embodiment, a chimeric polypeptide has formula (II):

(NH₂)SS-SBD-L-AA(COOH) (II)
wherein SS is an optional signal sequence (which is cleaved and removed from the chimeric polypeptide upon the secretion of the chimeric polypeptide by the recombinant host cell);
   SBD is a starch binding domain, a variant thereof, or a fragment thereof;
   L is an optional amino acid linker,
   AA is an alpha-amylase polypeptide, a variant thereof, or a fragment thereof and has alpha-amylase activity;
   (NH₂) indicates the amino terminus of the chimeric protein;
   (COOH) indicates the carboxyl terminus of the chimeric protein; and
   “-” is an amide linkage.

In formula (II), the carboxy terminus of optional SS is (directly or indirectly) associated with the amino terminus of SBD. The carboxy terminus of SBD is (directly or indirectly) associated with the amino terminus of optional L. The carboxy terminus of optional L is (directly or indirectly) associated with the amino terminus of AA. In one embodiment of the chimeric protein of formula (II), the carboxy terminus of SBD is directly associated with the amino terminus of the AA.

In some embodiments, a chimeric polypeptide is comprised of joining a polypeptide having alpha-amylase activity with a starch binding domain, and further has a signal sequence on the N-terminus of the chimeric polypeptide, wherein the chimeric polypeptide has formula (IA) or (IIA):

(NH₂)SS-AA-SBD(COOH) (IA) or

(NH₂)SS-SBD-AA(COOH) (IIA)

In formula (IA), the carboxy terminus of SS is (directly or indirectly) associated with the amino terminus of AA. The carboxy terminus of AA is (directly or indirectly) associated with the amino terminus of SBD.

In formula (IIA), the carboxy terminus of SS is (directly or indirectly) associated with the amino terminus of SBD. The carboxy terminus of SBD is (directly or indirectly) associated with the amino terminus of AA.

In some embodiments, a chimeric polypeptide is comprised of joining a polypeptide having alpha-amylase activity with a starch binding domain using a linker, wherein the chimeric polypeptide has formula (IB) or (IIB):

(NH₂)AA-L-SBD(COOH) (IB) or

(NH₂)SBD-L-AA(COOH) (IIB)

In formula (IB), the carboxy terminus of AA is (directly or indirectly) associated with the amino terminus of L. The carboxy terminus of L is (directly or indirectly) associated with the amino terminus of SBD.

In formula (IIB), the carboxy terminus of SBD is (directly or indirectly) associated with the amino terminus of L. The carboxy terminus of L is (directly or indirectly) associated with the amino terminus of AA.

In some embodiments, a chimeric polypeptide is comprised of joining a polypeptide having alpha-amylase activity with a starch binding domain using a linker and further having a signal sequence on the N-terminus of the chimeric polypeptide, wherein the chimeric polypeptide has formula (IC) or (IIC):

(NH₂)SS-AA-L-SBD(COOH) (IC) or

(NH₂)SS-SBD-L-AA(COOH) (IIC)

In formula (IC), the carboxy terminus of SS is (directly or indirectly) associated with the amino terminus of AA. The carboxy terminus of AA is (directly or indirectly) associated with the amino terminus of L. The carboxy terminus of L is (directly or indirectly) associated with the amino terminus of SBD.

In formula (IIC), the carboxy terminus of SS is (directly or indirectly) associated with the amino terminus of SBD. The carboxy terminus of SBD is (directly or indirectly) associated with the amino terminus of L. The carboxy terminus of L is (directly or indirectly) associated with the amino terminus of AA.

In some embodiments, a chimeric polypeptide is comprised of joining a polypeptide having alpha-amylase activity with a starch binding domain and lacks a signal sequence and a linker, wherein the chimeric polypeptide has formula (ID) or (IID):

(NH₂)AA-SBD(COOH) (ID)

(NH₂)SBD-AA(COOH) (IID)

In formula (ID), the carboxy terminus of AA is (directly or indirectly) associated with the amino terminus of SBD. In formula (IID), the carboxy terminus of SBD is (directly or indirectly) associated with the amino terminus of AA.

In an embodiment, a chimeric polypeptide is comprised of 1) a polypeptide having alpha-amylase activity from the genus Bacillus and, in some instances, from the species B. amyloliquefaciens, in further instances, encoded by the amyE gene from B. amyloliquefaciens; and 2) a starch binding domain having affinity to starch that is derived from a polypeptide having glucoamylase activity from the genus Aspergillus, in some instances, from the species Aspergillus niger, in further instances, from an Aspergillus niger G1 glucoamylase. In another embodiment, a chimeric polypeptide has the nucleic acid sequence of SEQ ID NO: 4, and/or the amino acid sequence of SEQ ID NO: 5 or 8.

Still in the context of the present disclosure, the chimeric polypeptides having alpha-amylase activity include variants of the chimeric polypeptides, such as, variants of the chimeric polypeptides having the amino acid sequence of SEQ ID NO: 5 or 8. A variant comprises at least one amino acid difference (substitution or addition) when compared to, for example, the amino acid sequence of the chimeric polypeptide of SEQ ID NO: 5 or 8. The chimeric polypeptide variants do exhibit alpha-amylase activity. In an embodiment, the variant chimeric polypeptide exhibits at least 50%, 60%, 70%, 80%, 90%, 95%, 96%, 97%, 98% or 99% of the alpha-amylase activity of the amino acid sequence of SEQ ID NO: 5 or 8. The chimeric polypeptide variants also have at least 70%, 80%, 85%, 90%, 95%, 96%, 97%, 98% or 99% identity to the amino acid sequence of SEQ ID NO: 5 or 8. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The present disclosure also provide fragments of the chimeric polypeptides and chimeric polypeptide variants described herein. A fragment comprises at least one less amino acid residue when compared to the amino acid sequence of the chimeric polypeptide or variant and still possess the enzymatic activity of the full-length chimeric polypeptide. In an embodiment, the fragment of the chimeric polypeptide exhibits at least 50%, 60%, 70%, 80%, 90%, 95%, 96%, 97%, 98% or 99% of the alpha-amylase activity of the full-length amino acid of SEQ ID NO: 5 or 8. The chimeric polypeptide fragments can also have at least 70%, 80%, 85%, 90%, 95%, 96%, 97%, 98% or 99% identity to the amino acid sequence of SEQ ID NO: 5 or 8. The fragment can be, for example, a truncation of one or more amino acid residues at the amino-terminus, the carboxy terminus or both terminus of the chimeric polypeptide or variant. Alternatively or in combination, the fragment can be generated from removing one or more internal amino acid residues. In an embodiment, the chimeric polypeptide fragment has at least 100, 150, 200, 250, 300, 350, 400, 450, 500, 550, 600, 650 or more consecutive amino acids of the chimeric polypeptide or the variant.

### Polypeptides Having Alpha-Amylase Activity

The chimeric polypeptides of the present disclosure includes an alpha-amylase (AA) moiety. Polypeptides having alpha-amylase activity (also referred to as alpha-amylases; EC 3.2.1.1) are endo-acting enzymes capable of hydrolyzing starch to maltose and maltodextrins. Alpha-amylases are calcium metalloenzymes which are unable to function in the absence of calcium. By acting at random locations along the starch chain, alpha-amylases break down long-chain carbohydrates, ultimately yielding maltotriose and maltose from amylose, or maltose, glucose and “limit dextrin” from amylopectin. Alpha-amylase activity can be determined by various ways by the person skilled in the art. For example, the alpha-amylase activity of a polypeptide can be determined directly by measuring the amount of reducing sugars generated by the polypeptide in an assay in which raw (corn) starch is used as the starting material. The alpha-amylase activity of a polypeptide can be measured indirectly by measuring the amount of reducing sugars generated by the polypeptide in an assay in which gelatinized (corn) starch is used as the starting material.

In the context of the present disclosure, the polypeptides having alpha-amylase activity can be derived from a bacteria, for example, from the genus Bacillus and, in some instances, from the species B. amyloliquefaciens. The polypeptides having alpha-amylase activity can be encoded by the amyE gene from B. amyloliquefaciens or an amyE gene ortholog. One example of alpha-amylase polypeptide is the AMYE polypeptide (GenBank Accession Number: ABS72727). The AMYE polypeptide comprises a catalytic domain (defined by amino acid residues located at positions 58 to 358) and an Aamy C domain (defined by amino acid residues located at positions 394 to 467). The AMYE polypeptide includes amino acid residues involved in the catalytic activity of the enzyme (e.g., active amino acid residues located at positions 99 to 100, 103 to 104, 143, 146, 171, 215, 217 to 218, 220 to 221, 249, 251, 253, 309 to 310, 314) as well as amino acid residues involved in binding calcium (e.g., amino acid residues located at position 142, 187 and 212). In an embodiment, the polypeptides having alpha-amylase activity comprises both a catalytic domain and an AamyC domain of the AMYE polypeptide as indicated above. In still another embodiment, the polypeptides having alpha-amylase activity have one or more (and in some embodiments all) the amino acid residues indicated above involved in the catalytic and calcium binding activity of the AMYE polypeptide.

In an embodiment, the polypeptides having alpha-amylase activity are encoded by the nucleotide sequence of SEQ ID NO: 1 or a nucleotide sequence encoding the amino acid sequence of SEQ ID NO: 2, 3 or 12. In another embodiment, the polypeptides having alpha-amylase activity comprises the amino acid sequence of SEQ ID NO: 2, 3 or 12.

In the context of the present disclosure, an “amyE gene ortholog” is understood to be a gene in a different species that evolved from a common ancestral gene by speciation. In the context of the present disclosure, an amyE ortholog retains the same function, e.g. it can act as an alpha-amylase. Known amyE gene orthologs include, but are not limited to those described at GenBank Accession numbers AGG59647.1 (B. subtilis), AHZ14317.1 (B. velezensis) and ACG63051.1 (Streptococcus equi), EFY01992 (Streptococcus dysgalactiae), EH168955 (Streptococcus ictaluri), EFF68324 (Butyvibrio crossotus), ADZ81868 (Clostidium lentocellum), AGX45116 (Clostridium saccharobutylicum), BAM49234 (Bacillus subtilis), ADP32662 (Bacillus atrophaeus), EFM08800 (Paenibacillus curdlanolytcus), EEP52889 (Clostridium butyricum) and COD81474 (Streptococcus pneumonia).

Still in the context of the present disclosure, the polypeptides having alpha-amylase activity include variants of the polypeptides, such as, variants of the alpha-amylases polypeptides of SEQ ID NO: 2, 3, or 12, or corresponding polypeptides encoded by a gene ortholog. A variant comprises at least one amino acid difference (substitution or addition) when compared to, for example, the amino acid sequence of the alpha-amylase polypeptide of SEQ ID NO: 2, 3, or 12. In an embodiment, the alpha-amylase variants comprise both the catalytic domain and the AamyC domain of the AMYE polypeptide indicated above. In still another embodiment, the alpha-amylase variants have one or more (and in some embodiments all) the amino acid residues indicated above involved in the catalytic and calcium binding activity of the AMYE polypeptide. The alpha-amylase variants do exhibit alpha-amylase activity. In an embodiment, the variant alpha-amylase exhibits at least 50%, 60%, 70%, 80%, 90%, 95%, 96%, 97%, 98% or 99% of the alpha-amylase activity of the amino acid of SEQ ID NO: 2, 3, or 12. The alpha-amylase variants also have at least 70%, 80%, 85%, 90%, 95%, 96%, 97%, 98% or 99% identity to the amino acid sequence of SEQ ID NO: 2, 3, 12. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, NY (1993): Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant alpha-amylases described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

A variant alpha-amylase can be also be a conservative variant or an allelic variant. As used herein, a conservative variant refers to alterations in the amino acid sequence that do not adversely affect the biological functions of the alpha amylase (e.g., hydrolysis of starch). A substitution, insertion or deletion is said to adversely affect the protein when the altered sequence prevents or disrupts a biological function associated with the alpha-amylase (e.g., the hydrolysis of starch into maltose and maltodextrins). For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the alpha-amylase.

The present disclosure also provide fragments of the alpha-amylases polypeptides and alpha-amylase variants described herein. A fragment comprises at least one less amino acid residue when compared to the amino acid sequence of the alpha-amylase polypeptide or variant and still possess the enzymatic activity of the full-length alpha-amylase. In an embodiment, the fragment of the alpha-amylase exhibits at least 50%, 60%, 70%, 80%, 90%, 95%, 96%, 97%, 98% or 99% of the alpha-amylase activity of the full-length amino acid of SEQ ID NO: 2, 3, or 12. In an embodiment, the alpha-amylase fragments comprises both the catalytic domain and the AamyC domain of the AMYE polypeptide as indicated above. In still another embodiment, the alpha-amylase fragment has one or more (and in some embodiments all) the amino acid residues indicated above involved in the catalytic and calcium binding activity of the AMYE polypeptide. The alpha-amylase fragments can also have at least 70%, 80%, 85%, 90%, 95%, 96%, 97%, 98% or 99% identity to the amino acid sequence of SEQ ID NO: 2, 3, or 12. The fragment can be, for example, a truncation of one or more amino acid residues at the amino-terminus, the carboxy terminus or both terminus of the alpha-amylase polypeptide or variant. Alternatively or in combination, the fragment can be generated from removing one or more internal amino acid residues. In an embodiment, the alpha-amylase fragment has at least 100, 150, 200, 250, 300, 350, 400, 450, 500, 550, 600, 650 or more consecutive amino acids of the alpha-amylase polypeptide or the variant.

Starch Binding Domain Moiety Starch binding domain (SBD) is a protein domain having carbohydrate-binding activity, and can bind to, for example, a starch molecule (e.g., raw starch). A starch binding domain can be found in carbohydrate-active enzyme, such as glucoamylases. The starch binding domain facilitates the activity of the enzyme having this protein domain, by providing or increasing binding affinity for the substrate molecule. As used herein, “binding affinity” refers to the strength of the binding interaction between a biomolecule (e.g. an enzyme) to its ligand or substrate (e.g. a starch molecule). Binding occurs by intermolecular forces, such as ionic bonds, hydrogen bonds and Van der Waals forces. In general, high-affinity ligand binding results from greater intermolecular force between the ligand and its biomolecule while low-affinity ligand binding involves less intermolecular force between the ligand and its biomolecule. In general, high-affinity binding results in a higher degree of occupancy for the ligand at its biomolecule binding site than is the case for low-affinity binding. High-affinity binding of ligands to biomolecules is often physiologically important when some of the binding energy can be used to cause a conformational change in the biomolecule, resulting in altered behavior of an associated ion channel or enzyme. In an embodiment, the starch binding domain has high affinity to starch molecules.

Binding affinity can be expressed using dissociation constant (K_{D}) values. In an embodiment, the starch binding domain of the present disclosure exhibits a high affinity to starch and in some embodiments to raw starch.

In an embodiment, the starch binding domain having affinity to starch is derived from a polypeptide having glucoamylase activity. In the context of the present disclosure, the polypeptide having glucoamylase activity can be derived from a fungus, for example, from the genus Aspergillus and, in some instances, from the species Aspergillus niger. In an embodiment, the starch binding domain comprises an linker and is derived from an Aspergillus niger G1 glucoamylase and is provided, for example, as the amino acid sequence of SEQ ID NO: 7, a variant of the amino acid sequence of SEQ ID NO: 7 or a fragment of the amino acid sequence of SEQ ID NO: 7.

Still in the context of the present disclosure, the starch binding domain includes variants of the domain, such as, variants of the starch binding domain of SEQ ID NO: 7. A variant comprises at least one amino acid difference (substitution or addition) when compared to, for example, the amino acid sequence of the starch binding domain of SEQ ID NO: 7. The starch binding domain variants do exhibit affinity to starch, and preferably high affinity to starch. In an embodiment, the variant starch binding domain has at least 70%, 80%, 85%, 90%, 95%, 96%, 97%, 98% or 99% identity to the amino acid sequence of SEQ ID NO: 7. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The present disclosure also provide fragments of the starch binding domain and starch binding domain variants described herein. A fragment comprises at least one less amino acid residue when compared to the amino acid sequence of the starch binding domain or variant and still possess affinity to starch, and preferably high affinity to starch. In an embodiment, the fragment of the starch binding exhibits at least 50%, 60%, 70%, 80%, 90%, 95%, 96%, 97%, 98% or 99% of the starch binding domain of the full-length amino acid of SEQ ID NO: 7. The starch binding domain fragments can also have at least 70%, 80%, 85%, 90%, 95%, 96%, 97%, 98% or 99% identity to the amino acid sequence of SEQ ID NO: 7. The fragment can be, for example, a truncation of one or more amino acid residues at the amino-terminus, the carboxy terminus or both terminus of the starch binding domain or variant. Alternatively or in combination, the fragment can be generated from removing one or more internal amino acid residues. In an embodiment, the starch binding domain fragment has at least 10, 15, 20, 25, 30, 40, 50 or more consecutive amino acids of the starch binding domain or the variant, which is described in WO/2018/002360, the disclosure of which are incorporated herein by reference.

### Signal Sequence

In some embodiments, the chimeric polypeptides of the present disclosure include a signal sequence. As used herein, a “signal sequence” refers to a short amino acid sequence presented at the N-terminus of a newly synthesized polypeptide that are destined towards the secretory pathway. Signal sequences can be found on polypeptides that reside either inside certain organelles (the endoplasmic reticulum, golgi or endosomes), secreted from the cell, or inserted into most cellular membranes. In some cases where the polypeptide is secreted from the cell, the signal sequence is cleaved from the polypeptide, freeing the polypeptide for secretion from the cell. In an embodiment, the signal sequence of chimeric polypeptide the present disclosure is endogenous to the alpha-amylase (AA) polypeptide. In another embodiment, the signal sequence of chimeric polypeptide the present disclosure is heterologous to the alpha-amylase (AA) polypeptide and can be derived from, for example, a polypeptide known to be secreted from its host. In some embodiments, one or more signal sequences can be used.

In an embodiment of the chimeric polypeptides of the present disclosure, the chimeric polypeptides include a signal sequence on the N-terminus of the polypeptide, such as the chimeric polypeptide of formula (I), (II), (IA), (IIA), (IC), or (IIC) (and can have, for example, the amino acid sequence of SEQ ID NO: 5, a variant thereof or a fragment thereof). In other embodiments, the chimeric polypeptides of the present disclosure lack a signal sequence (and can have, for example, the amino acid sequence of SEQ ID NO: 8, a variant thereof or a fragment thereof). In yet other embodiments, the chimeric polypeptides of the present disclosure are derived from cleaving the signal sequences of polypeptides having a signal sequence. In an embodiment of the polypeptides of the present disclosure, the signal sequences has the amino acid sequence of SEQ ID NO: 6, a variant thereof or a fragment thereof.

It is possible to use a polypeptide having alpha-amylase activity which does not comprise its endogenous signal sequence, such as, for example, the amino acid sequence of SEQ ID NO: 3. In an embodiment, the nucleotide molecule encoding the polypeptide having alpha-amylase activity can include a signal sequence which is endogenous to the host cell expressing the nucleotide molecule. For example, when the host is S. cerevisiae, the nucleotide molecule encoding the polypeptide can include the signal sequence of a protein endogenously expressed in S. cerevisiae, such as the signal sequence of the invertase protein (SUC2 and having, for example, an amino acid sequence of SEQ ID NO: 13, a variant thereof or a fragment thereof), from the AGA2 protein (and have, for example, an amino acid sequence of SEQ ID NO: 14, a variant thereof or a fragment thereof). In still another embodiment, the polypeptide can include the signal sequence of a protein that is not natively expressed in S. cerevisiae (such as, for example, from an alpha-amylase protein expressed in Aspergillus terreus and having, for example, the amino acid sequence of SEQ ID NO: 15, a variant thereof or a fragment thereof). In an embodiment, the nucleotide molecule encoding the polypeptide having alpha-amylase activity includes a signal sequence and is provided as nucleotide sequence of SEQ ID NO: 1; and the polypeptide having the signal sequence is provided as amino acid sequence of SEQ ID NO: 2.

In some embodiments, the signal sequence is from the gene encoding the invertase protein (and can have, for example, the amino acid sequence of SEQ ID NO: 13, a variant thereof or a fragment thereof) or the AGA2 protein (and can have, for example, the amino acid sequence of SEQ ID NO: 14, a variant thereof or a fragment thereof). In some embodiment, the signal sequence can be derived from a fungus, for example, from the genus Aspergillus and, in some instances, from the species Aspergillus terreus. In an embodiment, the signal sequence is derived from Aspergillus terreus alpha-amylase and provided as the amino acid sequence of SEQ ID NO: 15. In the context of the present disclosure, the expression “functional variant of a signal sequence” refers to a nucleic acid sequence that has been substituted in at least one nucleic acid position when compared to the native signal sequence which retain the ability to direct the expression of the chimeric polypeptide outside the cell. In the context of the present disclosure, the expression “functional fragment of a signal sequence” refers to a shorter nucleic acid sequence than the native signal sequence which retain the ability to direct the expression of the chimeric polypeptide outside the cell.

In some embodiments, a recombinant host cell has a heterologous nucleic acid molecule which includes a coding sequence for one or a combination of signal sequence(s) allowing the export of the heterologous chimeric polypeptide outside the yeast host cell's wall. The signal sequence can simply be added to the nucleic acid molecule (usually in frame with the sequence encoding the heterologous chimeric polypeptide) or replace the signal sequence already present in the heterologous chimeric polypeptide. The signal sequence can be native or heterologous to the nucleic acid sequence encoding the heterologous chimeric polypeptide or its corresponding chimera. In some embodiments, one or more signal sequences can be used.

### Amino Acid Linker

In some embodiments, the chimeric polypeptides of the present disclosure can include an amino acid linker. As used herein, the term “linker” refers to a short peptide sequences that is used to connect between two protein domains, two polypeptides, or a polypeptide and a protein domain. Linkers are often composed of flexible residues like glycine and serine so that the adjacent protein domains or polypeptides are free to move relative to one another. Longer linkers are used when it is necessary to ensure that two adjacent domains do not sterically interfere with one another. In some embodiments, the linkers of the present disclosure are intended such that the polypeptide having the alpha-amylase activity and the starch binding domain do not sterically interfere with one another.

In an embodiment of the chimeric polypeptides of the present disclosure, the chimeric polypeptides has an amino acid linker linking the polypeptide having alpha-amylase activity and the starch binding domain, such as the chimeric polypeptide of formula (I), (II), (IB), (IIB), (IC), or (IIC). In other embodiments, the chimeric polypeptides of the present disclosure lack a linker.

In some embodiments of the chimeric polypeptides of the present disclosure, the linker can be derived from a fungus, for example, from the genus Aspergillus and, in some instances, from the species Aspergillus niger. In an embodiment, the linker is derived from an Aspergillus niger G1 glucoamylase and provided as the amino acid sequence of SEQ ID NO: 16, a variant thereof or a fragment thereof. In another embodiment, the linker is derived from an Aspergillus niger GA and provided as the amino acid sequence of SEQ ID NO: 17, a variant thereof or a fragment thereof. In some embodiments, the linker is provided as the amino acid sequence of SEQ ID NO: 18, 19, 20, 21, 22, 23, 24, variants thereof, or fragments thereof.

In the context of the present disclosure, the expression “functional variant of a linker” refers to a nucleic acid sequence that has been substituted in at least one nucleic acid position when compared to the native linker which retain the ability to link the starch binding domain to the polypeptide having amylase activity of a chimeric polypeptide. In the context of the present disclosure, the expression “functional fragment of a linker” refers to a shorter nucleic acid sequence than the native signal sequence which retain the ability to link the starch binding domain to the polypeptide having amylase activity of a chimeric polypeptide.

### Polypeptides Having Glucoamylase Activity

The chimeric polypeptides of the present disclosure can be used in combination with a glucoamylase. Polypeptides having glucoamylase activity (also referred to as glucoamylases) are exo-acting enzymes capable of terminally hydrolyzing starch to glucose. Glucoamylase activity can be determined by various ways by the person skilled in the art. For example, the glucoamylase activity of a polypeptide can be determined directly by measuring the amount of reducing sugars generated by the polypeptide in an assay in which raw or gelatinized (corn) starch is used as the starting material.

In the context of the present disclosure, the polypeptides having glucoamylase activity can be derived from a yeast, for example, from the genus Saccharomycopsis and, in some instances, from the species S. fibuligera. The polypeptides having glucoamylase activity can be encoded by the glu0111 gene from S. fibuligera or a glu0111 gene ortholog. An embodiment of glucoamylase polypeptide of the present disclosure is the GLU0111 polypeptide (GenBank Accession Number: CAC83969.1). The GLU0111 polypeptide includes the following amino acids (or correspond to the following amino acids) which are associated with glucoamylase include, but are not limited to amino acids located at positions 41, 237, 470, 473, 479, 485, 487 of SEQ ID NO: 9. It is possible to use a polypeptide which does not comprise its endogenous signal sequence. In an embodiment, the polypeptides having glucoamylase activity include glucoamylases polypeptide comprising the amino acid sequence of SEQ ID NO: 9 or 11.

In the context of the present disclosure, a “glu0111 gene ortholog” is understood to be a gene in a different species that evolved from a common ancestral gene by speciation. In the context of the present disclosure, a glu0111 ortholog retains the same function, e.g. it can act as a glucoamylase. Glu0111 gene orthologs includes but are not limited to, the nucleic acid sequence of GenBank Accession Number XP_003677629.1 (Naumovozyma castellii) XP_003685231.1 (Tetrapisispora phaffii), XP_455264.1 (Kluyveromyces lactis), XP_446481.1 (Candida glabrata), EER33360.1 (Candida tropicalis), EEQ36251.1 (Clavispora lusitaniae), ABN68429.2 (Scheffersomyces stipitis), AAS51695.2 (Eremothecium gossypii), EDK43905.1 (Lodderomyces elongisporus), XP_002555474.1 (Lachancea themotolerans), EDK37808.2 (Pichia guilliermondii), CAA86282 (Saccharomyces cerevisiae), XP_003680486.1 (Torulaspora delbrueckii), XP_503574.1 (Yarrowia lipolytica), XP_002496552.1 (Zygosaccharomyces rouxii), CAX42655.1 (Candida dubliniensis), XP_002494017.1 (Komagataella pastoris) and AET38805.1 (Eremothecum cymbalariae).

Still in the context of the present disclosure, the polypeptides having glucoamylase activity include variants of the glucoamylases polypeptides of SEQ ID NO: 9 or 11 (also referred to herein as glucoamylase variants). A variant comprises at least one amino acid difference (substitution or addition) when compared to the amino acid sequence of the glucoamylase polypeptide of SEQ ID NO: 9 or 11. The glucoamylase variants do exhibit glucoamylase activity. In an embodiment, the variant glucoamylase exhibits at least 50%, 60%, 70%, 80%, 90%, 95%, 96%, 97%, 98% or 99% of the glucoamylase activity of the amino acid of SEQ ID NO: 9 or 11. The glucoamylase variants also have at least 70%, 80%, 85%, 90%, 95%, 96%, 97%, 98% or 99% identity to the amino acid sequence of SEQ ID NO: 9 or 11. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant glucoamylases described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

A variant glucoamylase can also be a conservative variant or an allelic variant. As used herein, a conservative variant refers to alterations in the amino acid sequence that do not adversely affect the biological functions of the glucoamylase. A substitution, insertion or deletion is said to adversely affect the protein when the altered sequence prevents or disrupts a biological function associated with the glucoamylase (e.g., the hydrolysis of starch into glucose). For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the glucoamylase.

In an embodiment, a glucoamylase variant has the amino acid sequence of SEQ ID NO: 10. The glucoamylase of SEQ ID NO: 9 and the glucoamylase variant of SEQ ID NO: 10 are described in WO/2018/002360, the disclosure of which are incorporated herein by reference.

The present disclosure also provide fragments of the glucoamylases polypeptides and glucoamylase variants described herein. A fragment comprises at least one less amino acid residue when compared to the amino acid sequence of the glucoamylase polypeptide or variant and still possess the enzymatic activity of the full-length glucoamylase. In an embodiment, the glucoamylase fragment exhibits at least 50%, 60%, 70%, 80%, 90%, 95%, 96%, 97%, 98% or 99% of the full-length glucoamylase of the amino acid of SEQ ID NO: 9, 10, or 11. The glucoamylase fragments can also have at least 70%, 80%, 85%, 90%, 95%, 96%, 97%, 98% or 99% identity to the amino acid sequence of SEQ ID NO: 9, 10, or 11. The fragment can be, for example, a truncation of one or more amino acid residues at the amino-terminus, the carboxy terminus or both termini of the glucoamylase polypeptide or variant. Alternatively or in combination, the fragment can be generated from removing one or more internal amino acid residues. In an embodiment, the glucoamylase fragment has at least 100, 150, 200, 250, 300, 350, 400, 450, 500 or more consecutive amino acids of the glucoamylase polypeptide or the variant.

Embodiments of polypeptides having glucoamylase activity have been also been described in PCT/US2012/032443 (published under WO/2012/138942) and PCT/US2011/039192 (published under WO/2011/153516) can also be used in the context of the present disclosure.

The polypeptides having glucoamylase activity, their fragments and their variants exhibit enzymatic activity towards raw starch. The GLU0111 polypeptide presented herein as well as glucomylases from Rhizopus oryzae and Corticium rolfsiiare are known to exhibit enzymatic activity towards raw starch.

### Methods of Making and Providing Chimeric Polypeptides

The chimeric polypeptide having alpha-amylase activity can be provided in a (substantially) purified or isolated form. As used in the context of the present disclosure, the expressions “purified form” or “isolated form” refers to the fact that the chimeric polypeptides have been physically dissociated from at least one components required for their production (such as, for example, a host cell or a host cell fragment). A purified form of the polypeptide of the present disclosure can be a cellular extract of a host cell expressing the polypeptide being enriched for the polypeptide of interest (either through positive or negative selection). The expressions “substantially purified form” or “substantially isolated” refer to the fact that the polypeptides have been physically dissociated from the majority of components required for their production (including, but not limited to, components of the recombinant yeast host cells). In an embodiment, a polypeptide in a substantially purified form is at least 90%, 95%, 96%, 97%, 98% or 99% pure.

The chimeric polypeptides having alpha-amylase activity are recombinant polypeptides. As used in the context of the present disclosure, the expression “recombinant form” refers to the fact that the polypeptides have been produced by recombinant DNA technology using genetic engineering to express the polypeptides in the recombinant (yeast) host cell.

The polypeptides described herein can independently be provided in a purified form or expressed in a recombinant host cell (e.g., the same or different recombinant host cells). The present disclosure concerns recombinant yeast host cells that have been genetically engineered.

The recombinant yeast host cells of the present disclosure include an heterologous nucleic acid molecule intended to allow the expression of (e.g., encode) one or more chimeric polypeptides and optionally one or more heterologous polypeptides. The genetic modification(s) is(are) aimed at increasing the expression of a specific targeted gene (which is considered heterologous to the yeast host cell) and can be made in one or multiple (e.g., 1, 2, 3, 4, 5, 6, 7, 8 or more) genetic locations. In the context of the present disclosure, when recombinant yeast cell is qualified as being “genetically engineered”, it is understood to mean that it has been manipulated to add at least one or more heterologous or exogenous nucleic acid residue. In some embodiments, the one or more nucleic acid residues that are added can be derived from an heterologous cell or the recombinant host cell itself. In the latter scenario, the nucleic acid residue(s) is (are) added at one or more genomic location which is different than the native genomic location. The genetic manipulations did not occur in nature and are the results of in vitro manipulations of the yeast.

When expressed in a recombinant host, the polypeptides described herein are encoded on one or more heterologous nucleic acid molecule. The heterologous nucleic acid molecule present in the recombinant host cell can be integrated in the host cell's genome. The term “integrated” as used herein refers to genetic elements that are placed, through molecular biology techniques, into the genome of a host cell. For example, genetic elements can be placed into the chromosomes of the host cell as opposed to in a vector such as a plasmid carried by the host cell. Methods for integrating genetic elements into the genome of a host cell are well known in the art and include homologous recombination. The heterologous nucleic acid molecule can be present in one or more copies (e.g., 2, 3, 4, 5, 6, 7, 8 or even more copies) in the yeast host cell's genome. Alternatively, the heterologous nucleic acid molecule can be independently replicating from the yeast's genome. In such embodiment, the nucleic acid molecule can be stable and self-replicating.

In the context of the present disclosure, the recombinant host cell can be a recombinant yeast host cell. Suitable recombinant yeast host cells can be, for example, from the genus Saccharomyces, Kluyveromyces, Arxula, Debaryomyces, Candida, Pichia, Phaffia, Schizosaccharomyces, Hansenula, Kloeckera, Schwanniomyces or Yarrowia. Suitable yeast species can include, for example, S. cerevisiae, S. bulderi, S. bametti, S. exiguus, S. uvarum, S. diastaticus, K. lactis, K. marxianus or K. fragilis. In some embodiments, the recombinant yeast host cell is selected from the group consisting of Saccharomyces cerevisiae, Schizzosaccharomyces pombe, Candida albicans, Pichia pastors, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In some embodiments, the recombinant yeast host cell is Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe or Schwanniomyces occidentalis. In some embodiment, the recombinant host cell can be an oleaginous yeast cell. For example, the recombinant oleaginous yeast host cell can be from the genera Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiments, the recombinant host cell can be an oleaginous microalgae host cell (e.g., for example, from the genera Thraustochytrium or Schizochytrium). In an embodiment, the recombinant yeast host cell is from the genus Saccharomyces and, in some embodiments, from the species Saccharomyces cerevisiae. In one particular embodiment, the recombinant yeast host cell is Saccharomyces cerevisiae.

One of the genetic modification that can be introduced into the recombinant host is the introduction of one or more of an heterologous nucleic acid molecule encoding a chimeric polypeptide (such as, for example, the chimeric polypeptides having alpha-amylase activity as described herein).

In a first embodiment, the recombinant host cell comprise a first genetic modification (e.g., a first heterologous nucleic acid molecule) allowing the recombinant expression of the chimeric polypeptide having alpha-amylase activity. In such embodiment, an heterologous nucleic acid molecule encoding the chimeric polypeptide having alpha-amylase activity can be introduced in the recombinant host to express the polypeptide having alpha-amylase activity. The expression of the chimeric polypeptide having alpha-amylase activity can be constitutive or induced.

The recombinant host cell comprising the first genetic modification can also include a further (second) genetic modification for reducing the production of one or more native enzymes that function to produce glycerol or regulate glycerol synthesis, for allowing the production of the second polypeptide having glucoamylase activity and/or for reducing the production of one or more native enzymes that function to catabolize formate. Alternatively, the recombinant host cell comprising the first genetic modification be used in combination with a further recombinant host cell which includes a further (second) genetic modification for reducing the production of one or more native enzymes that function to produce glycerol or regulate glycerol synthesis, for allowing the production of the second polypeptide having glucoamylase activity and/or for reducing the production of one or more native enzymes that function to catabolize formate.

As used in the context of the present disclosure, the expression “reducing the production of one or more native enzymes that function to produce glycerol or regulate glycerol synthesis” refers to a genetic modification which limits or impedes the expression of genes associated with one or more native polypeptides (in some embodiments enzymes) that function to produce glycerol or regulate glycerol synthesis, when compared to a corresponding host strain which does not bear the second genetic modification. In some instances, the second genetic modification reduces but still allows the production of one or more native polypeptides that function to produce glycerol or regulate glycerol synthesis. In other instances, the second genetic modification inhibits the production of one or more native enzymes that function to produce glycerol or regulate glycerol synthesis. In some embodiments, the recombinant host cells bear a plurality of second genetic modifications, wherein at least one reduces the production of one or more native polypeptides and at least another inhibits the production of one or more native polypeptides.

Alternatively, the recombinant host cell comprising the first genetic modification can also exclude a further (second) genetic modification for reducing the production of one or more native enzymes that function to produce glycerol or regulate glycerol synthesis, for allowing the production of the second polypeptide having glucoamylase activity and/or for reducing the production of one or more native enzymes that function to catabolize formate. In such embodiment, the recombinant host cell can be combined with a further (second) recombinant yeast host cells comprising the further (second) genetic modification.

As used in the context of the present disclosure, the expression “reducing the production of one or more native enzymes that function to produce glycerol or regulate glycerol synthesis” refers to a genetic modification which limits or impedes the expression of genes associated with one or more native polypeptides (in some embodiments enzymes) that function to produce glycerol or regulate glycerol synthesis, when compared to a corresponding host strain which does not bear the genetic modification. In some instances, the genetic modification reduces but still allows the production of one or more native polypeptides that function to produce glycerol or regulate glycerol synthesis. In other instances, the genetic modification inhibits the production of one or more native enzymes that function to produce glycerol or regulate glycerol synthesis. In some embodiments, the recombinant host cells bear a plurality of second genetic modifications, wherein at least one reduces the production of one or more native polypeptides and at least another inhibits the production of one or more native polypeptides.

As used in the context of the present disclosure, the expression “native polypeptides that function to produce glycerol or regulate glycerol synthesis” refers to polypeptides which are endogenously found in the recombinant host cell. Native enzymes that function to produce glycerol include, but are not limited to, the GPD1 and the GPD2 polypeptide (also referred to as GPD1 and GPD2 respectively). Native enzymes that function to regulate glycerol synthesis include, but are not limited to, the FPS1 polypeptide. In an embodiment, the recombinant host cell bears a genetic modification in at least one of the gpd1 gene (encoding the GPD1 polypeptide), the gpd2 gene (encoding the GPD2 polypeptide), the fps1 gene (encoding the FPS1 polypeptide) or orthologs thereof. In another embodiment, the fermenting yeast cell bears a genetic modification in at least two of the gpd1 gene (encoding the GPD1 polypeptide), the gpd2 gene (encoding the GPD2 polypeptide), the fps1 gene (encoding the FPS1 polypeptide) or orthologs thereof. In still another embodiment, the recombinant yeast host cell bears a genetic modification in each of the gpd1 gene (encoding the GPD1 polypeptide), the gpd2 gene (encoding the GPD2 polypeptide) and the fps1 gene (encoding the FPS1 polypeptide) or orthologs thereof. Examples of recombinant yeast host cells bearing such genetic modification(s) leading to the reduction in the production of one or more native enzymes that function to produce glycerol or regulate glycerol synthesis are described in WO 2012/138942. Preferably, the fermenting yeast cell has a genetic modification (such as a genetic deletion or insertion) only in one enzyme that functions to produce glycerol, in the gpd2 gene, which would cause the host cell to have a knocked-out gpd2 gene. In some embodiments, the fermenting yeast cell can have a genetic modification in the gpd1 gene, the gpd2 gene and the fps1 gene resulting is a recombinant host cell being knock-out for the gpd1 gene, the gpd2 gene and the fps1 gene.

As used in the context of the present disclosure, the expression “native polypeptides that function to catabolize formate” refers to polypeptides which are endogenously found in the fermenting yeast cell. Native enzymes that function to catabolize formate include, but are not limited to, the FDH1 and the FDH2 polypeptides (also referred to as FDH1 and FDH2 respectively). In an embodiment, the fermenting yeast cell bears a genetic modification in at least one of the fdh1 gene (encoding the FDH1 polypeptide), the fdh2 gene (encoding the FDH2 polypeptide) or orthologs thereof. In another embodiment, the fermenting yeast cell bears genetic modifications in both the fdh1 gene (encoding the FDH1 polypeptide) and the fdh2 gene (encoding the FDH2 polypeptide) or orthologs thereof. Examples of fermenting yeast cells bearing such genetic modification(s) leading to the reduction in the production of one or more native enzymes that function to catabolize formate are described in WO 2012/138942. Preferably, the fermenting yeast cell has genetic modifications (such as a genetic deletion or insertion) in the fdh1 gene and in the fdh2 gene which would cause the host cell to have knocked-out fdh1 and fdh2 genes.

In an embodiment, the recombinant fermenting yeast host cell includes a genetic modification does achieve higher pyruvate formate lyase activity in the recombinant or the further yeast host cell. This increase in pyruvate formate lyase activity is relative to a corresponding native yeast host cell which does not include the first genetic modification. As used in the context of the present disclosure, the term “pyruvate formate lyase” or “PFL” refers to an enzyme (EC 2.3.1.54) also known as formate C-acetytransferase, pyruvate formate-lyase, pyruvic formate-lyase and formate acetyltransferase. Pyruvate formate lyases are capable of catalyzing the conversion of coenzyme A (CoA) and pyruvate into acetyl-CoA and formate. In some embodiments, the pyruvate formate lyase activity may be increased by expressing an heterologous pyruvate formate lyase activating enzyme and/or a pyruvate formate lyase enzymate (such as, for example PFLA and/or PFLB).

In the context of the present disclosure, the genetic modification can include the introduction of an heterologous nucleic acid molecule encoding a pyruvate formate lyase activating enzyme and/or a puryvate formate lyase enzyme, such as PFLA. Embodiments of the pyruvate formate lyase activating enzyme and of PFLA can be derived, without limitation, from the following (the number in brackets correspond to the Gene ID number): Escherichia coli (MG1655945517), Shewanella oneidensis (1706020), Bifidobacterium longum (1022452), Mycobacterium bovis (32287203), Haemophilus parasuis (7277998), Mannheimia haemolytica (15341817), Vibrio vulnificus (33955434), Cronobacter sakazakii (29456271), Vibrio alginolyticus (31649536), Pasteurela multocida (29388611), Aggregatibacter actinomycetemcomitans (31673701), Actinobacillus suis (34291363), Finegoldia magna (34165045), Zymomonas mobilis subsp. mobilis (3073423), Vibrio tubiashii (23444968), Gallibacterium anatis (10563639), Actinobacillus pleuropneumoniae serovar (4849949), Ruminiclostdium themiocellum (35805539), Cylindrospemiopsis raciborskii (34474378), Lactococcus garvieae (34204939), Bacillus cytotoxicus (33895780), Providencia stuartii (31518098), Pantoes ananatis (31510290), Teredinibacter tumerae (29648846), Morganella morganii subsp. morgani (14670737), Vibrio anguillarum (77510775106), Dickeya dadantii (39379733484), Xenorhabdus bovienid (8830449), Edwardsiela ictalu (7959196), Proteus mirabilis (6801040), Rahnella aquatilis (34350771), Bacillus pseudomycoides (34214771), Vibrio alginolycus (29867350), Vibrio nigipulchritudo (29462895), Vibrio orientalis (25689084), Kosakonia sacchari (23844195), Serratia marcescens subsp. marcescens (23387394), Shewanella batica (11772864), Vibrio vulnificus (2625152), Streptomyces acidiscabies (33082227), Streptomyces davaonensis (31227069), Streptomyces scabiei (24308152), Volvox carteri f. nagaensis (9616877), Vibrio breoganii (35839746), Vibrio mediterranei (34766273), Fibrobacter succinogenes subsp. succinogenes (34755395), Enterococcus gilvus (34360882), Akkermansia muciniphila (34173806), Enterobacter hormaechei subsp. Steigerwaltii (34153767), Dickeya zeae (33924935), Enterobacter sp. (32442159), Serratia odoifera (31794665), Vibrio crassostreae (31641425), Selenomonas ruminantium subsp. lachlytica (31522409), Fusobacterium necrophorum subsp. funduliforme (31520833), Bacteroides uniformis (31507008), Haemophilus somnus (233631487328), Rodentibacter pneumotropicus (31211548), Pectobacterium carotovorum subsp. carotovorum (29706463), Eikenella corrodens (29689753), Bacillus thuringiensis (29685036), Streptomyces rimosus subsp. Rimosus (29531909), Vibrio fluvialis (29387180), Klebsiella oxytoca (29377541), Parageobacillus thermoglucosidans (29237437), Aeromonas veroni (28678409), Clostridium innocuum (26150741), Neissera mucosa (25047077), Citrobacter feundii (23337507), Clostrdium bolteae (23114831), Vibrio tasmaniensis (7160642), Aeromonas salmonicida subsp. samonicida (4995006), Escherichia coli 0157:H7 str. Sakai (917728), Escherichia coli 083:H1 str. (12877392), Yersinia pestis (11742220), Clostridioides difficile (4915332), Vibrio fischeri (3278678), Vibrio parahaemolyticus (1188496), Vibrio corallfilyticus (29561946), Kosakonia cowanhi (35808238), Yersinia ruckeri (29469535), Gardnerella vaginalis (99041930), Listeria fleischmannii subsp. Coloradonensis (34329629), Photobacterium kishitani (31588205), Aggregatibacter actinomycetemcomitans (29932581), Bacteroides caccae (36116123), Vibrio toranzoniae (34373279), Providencia alcalifaciens (34346411), Edwardsiella anguillarum (33937991), Lonsdalea quercina subsp. Quercina (33074607), Pantoea septica (32455521), Butyrivibdo proteoclasticus (31781353), Photorhabdus temperata subsp. Thracensis (29598129), Dickeya solani (23246485), Aeromonas hydrophila subsp. hydrophila (4489195), Vibrio cholerae O1 biovar El Tor str. (2613623), Serratfa rubidaea (32372861), Vibrio bivalvicida (32079218), Serratia liquefaciens (29904481), Giliamella apicola (29851437), Pluralibacter gergoviae (29488654), Escherichia coli 0104:H4 (13701423), Enterobacter aerogenes (10793245), Escherichia coli (7152373), Vibrio campbellii (5555486), Shigella dysenteriae (3795967), Bacillus thuringiensis serovar konkukian (2854507), Salmonella enterica subsp. enterica serovar Typhimurium (1252488), Bacillus anthracis (1087733), Shigella lexneri (1023839), Streptomyces giseoruber (32320335), Ruminococcus gnavus (35895414), Aeromonas fluvias (35843699), Streptomyces ossamyceticus (35815915), Xenorhabdus doucetise (34866557), Lactococcus piscium (34864314), Bacillus glycinifermentans (34773640), Photobacterium damselae subsp. Damselae 34509297, Streptomyces venezuelae 34035779, Shewanella algae (34011413), Neisseria sicca (33952518), Chania multitudinisentens (32575347), Kitasatospora purpeofusca (32375714), Serrata fonticola (32345867), Aeromonas enteropelogenes (32325051), Micromonospora aurantiaca (32162988), Moritella viscosa (31933483), Yersinia aldovee (31912331), Leclercia adecarboxylata (31868528), Salinivibrio costicola subsp. costicola (31850688), Aggregatibacter aphrophilus (31611082), Photobacterium leiognathi (31590325), Streptomyces canus (31293262), Pantoea dispersa (29923491), Pantoea rwandensis (29806428), Paenibacllus borealis (29548601), Alivibrio wodanis (28541257), Streptomyces virginiae (23221817), Escherichis coli (7158493), Mycobacterium tuberculosis (887973), Streptococcus mutans (1028925), Streptococcus cristatus (29901602), Enterococcus hirae (13176624), Bacillus licheniformis (3031413), Chromobacterium violaceum (24949178), Parabacteroides distasonis (5308542), Bacteroides vulgatus (5303840), Faecalibacterium prausnitzii (34753201), Melissococcus plutonius (34410474), Streptococcus gallolyticus subsp. gallolyticus (34397064), Enterococcus melodoratus (34355146), Bacteroides oleiciplenus (32503668), Listeda monocytogenes (985766), Enterococcus faecalis (1200510), Campylobacter jejuni subsp. jejuni (905864), Lactobacillus plantarum (1063963), Yersinia enterocolitica subsp. enterocolitica (4713333), Streptococcus equinus (33961143), Macrococcus canis (35294771), Streptococcus sanguinis (4807186), Lactobacillus salivarus (3978441), Lactococcus lacis subsp. lactis (1115478), Enterococcus faecium (12999835), Clostridium botulinum A (5184387), Clostrdium acetobutylicum (1117164), Bacillus thurngiensis serovar konkukian (2857050), Cryobacterium flavum (35899117), Enterovibrio norvegicus (35871749), Bacillus acidiceler (34874556), Prevotella intermedia (34516987), Pseudobutyrivibrio ruminis (34419801), Pseudovibrio ascidiaceicola (34149433), Corynebacterium coyleae (34026109), Lactobacillus curvatus (33994172), Cellulosimicrobium celulans (33980622), Lactobacilus agilis (33975995), Lactobacillus sakei (33973512), Staphylococcus simulans (32051953), Obesumbaterium proteus (29501324), Salmonella enterca subsp. entenca serovar Typhi (1247402), Streptococcus agalactiae (1014207), Streptococcus agalactiae (1013114), Legionella pneumophila subsp. pneumophila str. Philadelphia (119832735), Pyrococcus furiosus (1468475), Mannheimia haemolytica (15340992), Thalassiosira pseudonana (7444511), Thalassiosira pseudonana (7444510), Streptococcus thermophilus (31940129), Sulfolobus solfatancus (1454925), Streptococcus iniae (35765828), Streptococcus iniae (35764800), Bifidobactedum thermophilum (31839084), Bifidobacterium animalis subsp. lactis (29695452), Streptobacillus moniliformis (29673299), Thermogladius calderae (13013001), Streptococcus oralis subsp. tigurinus (31538096), Lactobacillus ruminis (29802671), Streptococcus parauberis (29752557), Bacteroides ovatus (29454036), Streptococcus gordonii str. Challis substr. CH1 (25052319), Clostridium botulinum B str. Eklund 17B (19963260), Thermococcus litoralis (16548368), Archaeoglobus sulfaticallidus (15392443), Ferrogobus placidus (8778929), Archaeoglobus profundus (8739370), Listena seeligeri serovar 1/2b (32488230), Bacillus thuringiensis (31632063), Rhodobacter capsulatus (31491679), Clostidium botulinum (29749009), Clostridium perfringens (29571530), Lactococcus garvieae (12478921), Proteus mirabilis (6799920), Lactobacillus animalis (32012274), Vibrio alginolyticus (29869205), Bacteroides thetaiotaomicron (31617701), Bacteroides thetaiotaomicron (31617140), Bacteroides cellulosilyticus (29608790), Bacteroides ovatus (29453452), Bacillus mycoides (29402181), Chlamydomonas reinhardtii (5726206), Fusobacterium periodonticum (35833538), Selenomonas flueggei (32477557), Selenomonas noxia (32475880), Anaerococcus hydrogenalis (32462628), Centipeda periodontii (32173931), Centipeda periodontii (32173899), Streptococcus thermophilus (31938326), Enterococcus durans (31916360), Fusobacterium nucleatum (31730399), Anaerostpes hadrus (31625694), Anaerostipes hadrus (31623667), Enterococcus haemoperoxidus (29838940), Gardnerela vaginalis (29692621), Streptococcus salivarius (29397526), Klebsiella oxytoca (29379245), Bifdobacterium breve (29241363), Actinomyces odontolyticus (25045153), Haemophilus ducreyi (24944624), Archaeoglobus fulgidus (24793671), Streptococcus uberis (24161511), Fusobacterium nucleatum subsp. animalis (23369066), Corynebacterium accolens (23249616), Archaeoglobus veneficus (10394332), Prevotella melaninogenica (9497682), Aeromonas salmonicida subsp. salmonicida (4997325), Pyrobaculum islandicum (4616932), Thermofilum pendens (4600420), Bifdobacterium adolescentis (4556560), Listeria monocytogenes (986485), Bindobacterium thermophilum (35776852), Methanothermobacter sp. CaT2 (24854111), Streptococcus pyogenes (901706), Exiguobacterium sibincum (31768748), Clostridioides difficile (4916015), Clostridioides difficile (4913022), Vibrio parahaemoyticus (1192264), Yersinia enterocolitica subsp. enterocolitica (4712948), Enterococcus cecorum (29475065), Bifidobacterium pseudolongum (34879480), Methanothermus feividus (9962832), Methanothermus fervidus (9962056), Corynebacterium simulans (29536891), Thermoproteus uzoniensis (10359872), Vulcanisseta distributa (9752274), Streptococcus mitis (8799048), Ferroglobus placidus (8778420), Streptococcus suis (8153745), Clostridium novyi (4541619), Streptococcus mutans (1029528), Thermosynechococcus elongatus (1010568), Chlorobium tepidum (1007539), Fusobacterium nucleatum subsp. nucleatum (993139), Streptococcus pneumoniae (933787), Clostridium baratii (31579258), Enterococcus mundtii (31547246), Prevotella ruminicola (31500814), Aeromonas hydrophila subsp. hydrophila (4490168), Aeromonas hydrophila subsp. hydrophila (4487541), Clostridium acetobutylicum (1117604), Chromobacterium subtsugae (31604683), Gilliamella apicola (29849369), Klebsiella pneumoniae subsp. pneumoniae (11846825), Enterobacter cloacae subsp. cloacae (9125235), Escherichia coli (7150298), Salmonella enterica subsp. enterica serovar Typhimurium (1252363), Salmonella enterica subsp. enterica serovar Typhi (1247322), Bacillus cereus (1202845), Bacteroides thetaotaomicron (1074343), Bacteroides thetaiotaomicron (1071815), Bacillus coagulans (29814250), Bacteroides cellulosilyticus (29610027), Bacillus anthracis (2850719), Monoraphidium neglectum (25735215), Monoraphidium neglectum (25727595), Alloscardovia omnicolens (35868062), Actinomyces neuii subsp. neuii (35867196), Acetoanaerobium sticklandii (35557713), Exiguobacterium undae (32084128), Paenibacillus pabuli (32034589), Paenibacillus etheri (32019864), Actinomyces oris (31655321), Vibrio alginolyticus (31651485), Brochothrx thermosphacta (29820407), Lactobacillus sakei subsp. sakei (29638315), Anoxybacillus gonensis (29574914), variants thereof as well as fragments thereof. In an embodiment, the PFLA protein is derived from the genus Bifidobacterium and in some embodiments from the species Bifidobacterium adolescentis. In an embodiment, the heterologous nucleic acid molecule encoding the PFLA protein is present in at least one, two, three, four, five or more copies in the recombinant yeast host cell. In still another embodiment, the heterologous nucleic acid molecule encoding the PFLA protein is present in no more than five, four, three, two or one copy/ies in the recombinant yeast host cell.

In the context of the present disclosure, the recombinant host cell has a genetic modification encoding a formate acetyltransferase enzyme and/or a puryvate formate lyase enzyme, such as PFLB. Embodiments of PFLB can be derived, without limitation, from the following (the number in brackets correspond to the Gene ID number): Escherichia coli (945514), Shewanella oneidensis (1170601), Actinobacillus suis (34292499), Finegoldia magna (34165044), Streptococcus cristatus (29901775), Enterococcus hirae (13176625), Bacillus (3031414), Providencia alcalifaciens (34345353), Lactococcus garvieae (34203444), Butyrivibrio proteoclastcus (31781354), Teredinibacter tumerae (29651613), Chromobacterium violaceum (24945652), Vibrio campbellii (5554880), Vibrio campbellii (5554796), Rahnella aquatilis HX2 (34351700), Serrada rubidaea (32375076), Kosakonia sacchari SP1 (23845740), Shewanella baltica (11772863), Streptomyces acidiscabies (33082309), Streptomyces davaonensis (31227068), Parabacteroides distasonis (5308541), Bacteroides vulgatus (5303841), Fibrobacter succinogenes subsp. succinogenes (34755392), Photobacterium damselae subsp. Damselae (34512678), Enterococcus gilvus (34361749), Enterococcus gilvus (34360863), Enterococcus malodoratus (34355213), Enterococcus malodoratus (34354022), Akkermansia muciniphila (34174913), Lactobacillus curvatus (33995135), Dickeya zeae (33924934), Bacteroides oleiciplenus (32502326), Micromonospora aurantiaca (32162989), Selenomonas ruminantium subsp. lactilytica (31522408), Fusobacterium necrophorum subsp. fundulibrme (31520832), Bacteroides uniformis (31507007), Streptomyces rimosus subsp. Rimosus (29531908), Clostdium innocuum (26150740), Haemophilus] ducreyi (24944556), Closridium boteae (23114829), Vibrio tasmaniensis (7160644), Aeromonas salmonicida subsp. salmonicida (4997718), Listeria monocytogenes (986171), Enterococcus faecalis (1200511), Lactobacillus plantarum (1064019), Vibrio fischeri (3278780), Lactobacillus sakei (33973511), Gardnerela vaginalis (9904192), Vibrio vulnificus (33954428), Vibrio toranzoniae (34373229), Anaerostpes hadrus (34240161), Edwardsiella anguillarum (33940299), Edwardsiella anguillarum (33937990), Lonsdalea quercina subsp. Quercina (33074710), Enterococcus faecium (12999834), Aeromonas hydrophila subsp. hydrophila (4489100), Clostridium acetobutylicum (1117163), Escherichia coli (7151395), Shigella dysentenae (3795966), Bacillus thuringiensis serovar konkukian (2856201), Salmonella enterica subsp. enteica serovar Typhimurium (1252491), Shigella flexneri (1023824), Streptomyces griseoruber (32320336), Cryobacterium flavum (35898977), Ruminococcus gnavus (35895748), Bacillus acidiceler (34874555), Lactococcus piscium (34864362), Vibrio mediterranei (34766270), Faecalibacterium prausnitzii (34753200), Prevotella intermedia (34516966), Photobacterium damselae subsp. Damselae (34509286), Pseudobutyrivibrio ruminis (34419894), Melissococcus plutonius (34408953), Streptococcus gallolyticus subsp. gallolyticus (34398704), Enterobacter hormaechei subsp. Steigerwalii (34155981), Enterobacter hormaechei subsp. Steigerwaltii (34152298), Streptomyces venezuelae (34036549), Shewanella algae (34009243), Lactobacillus agis (33976013), Streptococcus equinus (33961013), Neisseria sicca (33952517), Kitasatospora purpeofusca (32375782), Paenibacillus borealis (29549449), Vibrio fluvialis (29387150), Aliivibrio wodanis (28542465), Aliivibrio wodanis (28541256), Escherchia coli (7157421), Salmonella enterica subsp. enterca serovar Typhi (1247405), Yersinia pesis (1174224), Yersinia enterocolitica subsp. enterocolitica (4713334), Streptococcus suis (8155093), Escherichia coli (947854), Escherichia coli (946315), Escherchia coli (945513), Escherichia coli (948904), Escherichia coli (917731), Yersinia enterocolitica subsp. enterocolitica (4714349), variants thereof as well as fragments thereof. In an embodiment, the PFLB protein is derived from the genus Bifidobacterium and in some embodiments from the specifies Bifidobacterium adolescentis. In an embodiment, the heterologous nucleic acid molecule encoding the PFLB protein is present in at least one, two, three, four, five or more copies in the recombinant yeast host cell. In still another embodiment, the heterologous nucleic acid molecule encoding the PFLB protein is present in no more than five, four, three, two or one copy/ies in the recombinant yeast host cell.

In some embodiments, the recombinant host cell comprises a genetic modification for expressing a PFLA protein, a PFLB protein or a combination. In a specific embodiment, the recombinant host cell comprises a genetic modification for expressing a PFLA protein and a PFLB protein which can, in some embodiments, be provided on distinct heterologous nucleic acid molecules.

The recombinant host cell can also include additional genetic modifications to provide or increase its ability to transform acetyl-CoA into an alcohol such as ethanol. Alternatively or in combination, the recombinant host cell can bear one or more genetic modification for utilizing acetyl-CoA for example, by providing or increasing acetaldehyde and/or alcohol dehydrogenase activity. Acetyl-coA can be converted to an alcohol such as ethanol using first an acetaldehyde dehydrogenase and then an alcohol dehydrogenase. Acylating acetaldehyde dehydrogenases (E.C. 1.2.1.10) are known to catalyze the conversion of acetaldehyde into acetyl-coA in the presence of coA. Alcohol dehydrogenases (E.C. 1.1.1.1) are known to be able to catalyze the conversion of acetaldehyde into ethanol. The acetaldehyde dehydrogenase and alcohol dehydrogenase activity can be provided by a single protein (e.g., a bifunctional acetaldehyde/alcohol dehydrogenase) or by a combination of more than one protein (e.g., an acetaldehyde dehydrogenase and an alcohol dehydrogenase). In embodiments in which the acetaldehyde/alcohol dehydrogenase activity is provided by more than one protein, it may not be necessary to provide the combination of proteins in a recombinant form in the recombinant yeast host cell as the cell may have some pre-existing acetaldehyde or alcohol dehydrogenase activity. In such embodiments, the genetic modification can include providing one or more heterologous nucleic acid molecule encoding one or more of an heterologous acetaldehyde dehydrogenase (AADH), an heterologous alcohol dehydrogenase (ADH) and/or heterologous bifunctional acetylaldehyde/alcohol dehydrogenases (ADHE). For example, the genetic modification can comprise introducing an heterologous nucleic acid molecule encoding an acetaldehyde dehydrogenase. In another example, the genetic modification can comprise introducing an heterologous nucleic acid molecule encoding an alcohol dehydrogenase. In still another example, the genetic modification can comprise introducing at least two heterologous nucleic acid molecules, a first one encoding an heterologous acetaldehyde dehydrogenase and a second one encoding an heterologous alcohol dehydrogenase. In another embodiment, the genetic modification comprises introducing an heterologous nucleic acid encoding an heterologous bifunctional acetylaldehyde/alcohol dehydrogenases (AADH) such as those described in U.S. Pat. No. 8,956,851 and WO 2015/023989. Heterologous AADHs of the present disclosure include, but are not limited to, the ADHE polypeptides or a polypeptide encoded by an adhe gene ortholog.

The recombinant host cell can be further genetically modified to allow for the production of additional heterologous polypeptides. In an embodiment, the recombinant yeast host cell can be used for the production of an enzyme, and especially an enzyme involved in the cleavage or hydrolysis of its substrate (e.g., a lytic enzyme and, in some embodiments, a saccharolytic enzyme). In still another embodiment, the enzyme can be a glycoside hydrolase. In the context of the present disclosure, the term “glycoside hydrolase” refers to an enzyme involved in carbohydrate digestion, metabolism and/or hydrolysis, including amylases (other than those described above), cellulases, hemicellulases, cellulolytic and amylolytic accessory enzymes, inulinases, levanases, trehalases, pectinases, and pentose sugar utilizing enzymes. In another embodiment, the enzyme can be a protease. In the context of the present disclosure, the term “protease” refers to an enzyme involved in protein digestion, metabolism and/or hydrolysis. In yet another embodiment, the enzyme can be an esterase.

In the context of the present disclosure, the term “esterase” refers to an enzyme involved in the hydrolysis of an ester from an acid or an alcohol, including phosphatases such as phytases.

In order to make the recombinant yeast host cells, heterologous nucleic acid molecules (also referred to as expression cassettes) are made in vitro and introduced into the yeast host cell in order to allow the recombinant expression of the chimeric polypeptides described herein.

The heterologous nucleic acid molecules of the present disclosure comprise a coding region for the heterologous polypeptide, e.g., the chimeric polypeptides described herein. A DNA or RNA “coding region” is a DNA or RNA molecule (preferably a DNA molecule) which is transcribed and/or translated into a chimeric polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The nucleic acid molecules described herein can comprise transcriptional and/or translational control regions. “Transcriptional and translational control regions” are DNA regulatory regions, such as promoters, enhancers, terminators, and the like, that provide for the expression of a coding region in a host cell. In eukaryotic cells, polyadenylation signals are control regions.

In some embodiments, the heterologous nucleic acid molecules of the present disclosure include a promoter as well as a coding sequence for the chimeric polypeptides described herein. The heterologous nucleic acid sequence can also include a terminator. In the heterologous nucleic acid molecules of the present disclosure, the promoter and the terminator (when present) are operatively linked to the nucleic acid coding sequence of the chimeric polypeptide (including chimeric proteins comprising same), e.g., they control the expression and the termination of expression of the nucleic acid sequence of the chimeric polypeptide. The heterologous nucleic acid molecules of the present disclosure can also include a nucleic acid coding for a signal peptide, e.g., a short peptide sequence for exporting the chimeric polypeptide outside the host cell. When present, the nucleic acid sequence coding for the signal peptide is directly located upstream and is in frame with the nucleic acid sequence coding for the chimeric polypeptide.

In the heterologous nucleic acid molecule described herein, the promoter and the nucleic acid molecule coding for the heterologous polypeptide are operatively linked to one another.

In the context of the present disclosure, the expressions “operatively linked” or “operatively associated” refers to fact that the promoter is physically associated to the nucleotide acid molecule coding for the heterologous polypeptide in a manner that allows, under certain conditions, for expression of the heterologous protein from the nucleic acid molecule. In an embodiment, the promoter can be located upstream (5′) of the nucleic acid sequence coding for the heterologous protein. In still another embodiment, the promoter can be located downstream (3) of the nucleic acid sequence coding for the heterologous protein. In the context of the present disclosure, one or more than one promoter can be included in the heterologous nucleic acid molecule. When more than one promoter is included in the heterologous nucleic acid molecule, each of the promoters is operatively linked to the nucleic acid sequence coding for the heterologous protein. The promoters can be located, in view of the nucleic acid molecule coding for the heterologous protein, upstream, downstream as well as both upstream and downstream.

“Promoter” refers to a DNA fragment capable of controlling the expression of a coding sequence or functional RNA. The term “expression,” as used herein, refers to the transcription and stable accumulation of sense (mRNA) from the heterologous nucleic acid molecule described herein. Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of the polymerase.

The promoter can be native or heterologous to the nucleic acid molecule encoding the heterologous polypeptide. The promoter can be heterologous or derived from a strain being from the same genus or species as the recombinant host cell. In an embodiment, the promoter is derived from the same genus or species of the yeast host cell and the heterologous polypeptide is derived from a different genus than the host cell. The promoter can be a single promoter or a combination of different promoters.

In the present disclosure, promoters allowing or favoring the expression of the chimeric polypeptides during the propagation phase of the recombinant yeast host cells are preferred. Yeasts that are facultative anaerobes, are capable of respiratory reproduction under aerobic conditions and fermentative reproduction under anaerobic conditions. In many commercial applications, yeast are propagated under aerobic conditions to maximize the conversion of a substrate to biomass. Optionally, the biomass can be used in a subsequent fermentation under anaerobic conditions to produce a desired metabolite. In the context of the present disclosure, it is important that the promoter or combination of promoters present in the heterologous nucleic acid is/are capable of allowing the expression of the chimeric polypeptide during the propagation phase of the recombinant yeast host cell. This will allow the accumulation of the chimeric polypeptides associated with the recombinant yeast host cell prior to fermentation (if any). In some embodiments, the promoter allows the expression of the chimeric polypeptide during propagation, but not during fermentation (if any) of the recombinant yeast host cell.

The promoters can be native or heterologous to the heterologous gene encoding the heterologous protein. The promoters that can be included in the heterologous nucleic acid molecule can be constitutive or inducible. Inducible promoters include, but are not limited to glucose-regulated promoters (e.g., the promoter of the hxt7 gene (referred to as hxt7p); the promoter of the ctt1 gene (referred to as ctt1p), a functional variant or a functional fragment thereof; the promoter of the glo1 gene (referred to as glo1p), a functional variant or a functional fragment thereof; the promoter of the ygp1 gene (referred to as ygp1p), a functional variant or a functional fragment thereof; the promoter of the gsy2 gene (referred to as gsy2p, a functional variant or a functional fragment thereof), molasses-regulated promoters (e.g., the promoter of the mol1 gene (referred to as mol1p), a functional variant or a functional fragment thereof), heat shock-regulated promoters (e.g., the promoter of the glo1 gene (referred to as glo1p), a functional variant or a functional fragment thereof; the promoter of the sti1 gene (referred to as sti1p), a functional variant or a functional fragment thereof; the promoter of the ygp1 gene (referred to as ygp1p), a functional variant or a functional fragment thereof; the promoter of the gsy2 gene (referred to as gsy2p), a functional variant or a functional fragment thereof), oxidative stress response promoters (e.g., the promoter of the cup1 gene (referred to as cup1p), a functional variant or a functional fragment thereof; the promoter of the cit1 gene (referred to as cit1p), a functional variant or a functional fragment thereof; the promoter of the trx2 gene (referred to as trx2p), a functional variant or a functional fragment thereof; the promoter of the gpd1 gene (referred to as gpd1p), a functional variant or a functional fragment thereof; the promoter of the hsp12 gene (referred to as hsp12p), a functional variant or a functional fragment thereof), osmotic stress response promoters (e.g., the promoter of the ctt1 gene (referred to as ctt1p), a functional variant or a functional fragment thereof; the promoter of the glo1 gene (referred to as glo1p), a functional variant or a functional fragment thereof; the promoter of the gpd1 gene (referred to as gpd1p), a functional variant or a functional fragment thereof; the promoter of the ygp1 gene (referred to as ygp1p), a functional variant or a functional fragment thereof) and nitrogen-regulated promoters (e.g., the promoter of the ygp1 gene (referred to as ygp1p), a functional variant or a functional fragment thereof).

Promoters that can be included in the heterologous nucleic acid molecule of the present disclosure include, without limitation, the promoter of the tdh1 gene, of the hor7 gene, of the hsp150 gene, of the hxt7 gene, of the gpm1 gene, of the pgk1 gene and/or of the stl1 gene (referred to as stl1p, a functional variant or a functional fragment thereof). In an embodiment, the promoter is or comprises the tdh1p and/or the hor7p. In still another embodiment, the promoter comprises or consists essentially of the tdh1p and the hor7p. In a further embodiment, the promoter is the thd1p.

In the context of the present disclosure, the promoter controlling the expression of the heterologous polypeptide can be a constitutive promoter (such as, for example, tef2p (e.g., the promoter of the tef2 gene), cwp2p (e.g., the promoter of the cwp2 gene), ssa1p (e.g., the promoter of the ssa1 gene), eno1p (e.g., the promoter of the eno1 gene), hxk1 (e.g., the promoter of the hxk1 gene) and pgk1p (e.g., the promoter of the pgk1 gene). In some embodiment, the promoter is adh1p (e.g., the promoter of the adh1 gene). However, is some embodiments, it is preferable to limit the expression of the polypeptide. As such, the promoter controlling the expression of the heterologous polypeptide can be an inducible or modulated promoters such as, for example, a glucose-regulated promoter (e.g., the promoter of the hxt7 gene (referred to as hxt7p)) or a sulfite-regulated promoter (e.g., the promoter of the gpd2 gene (referred to as gpd2p or the promoter of the fzf1 gene (referred to as the fzf1p)), the promoter of the ssu1 gene (referred to as ssu1p), the promoter of the ssu1-r gene (referred to as ssur1-rp). In an embodiment, the promoter is an anaerobic-regulated promoters, such as, for example tdh1p (e.g., the promoter of the tdh1 gene), pau5p (e.g., the promoter of the pau5 gene), hor7p (e.g., the promoter of the hor7 gene), adh1p (e.g., the promoter of the adh1 gene), tdh2p (e.g., the promoter of the tdh2 gene), tdh3p (e.g., the promoter of the tdh3 gene), gpd1p (e.g., the promoter of the gdp1 gene), cdc19p (e.g., the promoter of the cdc19 gene), eno2p (e.g., the promoter of the eno2 gene), pdc1p (e.g., the promoter of the pdc1 gene), hxt3p (e.g., the promoter of the hxt3 gene), dan1 (e.g., the promoter of the dan1 gene) and tpi1p (e.g., the promoter of the tpi1 gene).

One or more promoters can be used to allow the expression of each heterologous polypeptides in the recombinant yeast host cell. In the context of the present disclosure, the expression “functional fragment of a promoter” when used in combination to a promoter refers to a shorter nucleic acid sequence than the native promoter which retain the ability to control the expression of the nucleic acid sequence encoding the chimeric polypeptide during the propagation phase of the recombinant yeast host cells. Usually, functional fragments are either 5′ and/or 3′ truncation of one or more nucleic acid residue from the native promoter nucleic acid sequence.

In some embodiments, the nucleic acid molecules include a one or a combination of terminator sequence(s) to end the translation of the chimeric polypeptide. The terminator can be native or heterologous to the nucleic acid sequence encoding the chimeric polypeptide. In some embodiments, one or more terminators can be used. In some embodiments, the terminator comprises the terminator from is from the dit1 gene, from the idp1 gene, from the gpm1 gene, from the pma1 gene, from the tdh3 gene, from the hxt2 gene, from the adh3 gene, from the cycl gene, from the pgk1 gene and/or from the ira2 gene. In an embodiment, the terminator is derived from the dit1 gene. In another embodiment, the terminator comprises or is derived from the adh3 gene. In the context of the present disclosure, the expression “functional variant of a terminator” refers to a nucleic acid sequence that has been substituted in at least one nucleic acid position when compared to the native terminator which retain the ability to end the expression of the nucleic acid sequence coding for the heterologous protein or its corresponding chimera. In the context of the present disclosure, the expression “functional fragment of a terminator” refers to a shorter nucleic acid sequence than the native terminator which retain the ability to end the expression of the nucleic acid sequence coding for the heterologous protein or its corresponding chimera.

The heterologous nucleic acid molecule encoding the chimeric polypeptide, variant or fragment thereof can be integrated in the genome of the yeast host cell. The term “integrated” as used herein refers to genetic elements that are placed, through molecular biology techniques, into the genome of a host cell. For example, genetic elements can be placed into the chromosomes of the host cell as opposed to in a vector such as a plasmid carried by the host cell. Methods for integrating genetic elements into the genome of a host cell are well known in the art and include homologous recombination. The heterologous nucleic acid molecule can be present in one or more copies in the yeast host cell's genome. Alternatively, the heterologous nucleic acid molecule can be independently replicating from the yeast's genome. In such embodiment, the nucleic acid molecule can be stable and self-replicating.

The present disclosure also provides nucleic acid molecules for modifying the yeast host cell so as to allow the expression of the chimeric polypeptides, variants or fragments thereof. The nucleic acid molecule may be DNA (such as complementary DNA, synthetic DNA or genomic DNA) or RNA (which includes synthetic RNA) and can be provided in a single stranded (in either the sense or the antisense strand) or a double stranded form. The contemplated nucleic acid molecules can include alterations in the coding regions, non-coding regions, or both. Examples are nucleic acid molecule variants containing alterations which produce silent substitutions, additions, or deletions, but do not alter the properties or activities of the chimeric polypeptides, variants or fragments thereof.

In some embodiments, the nucleic acid molecules encoding the heterologous polypeptides, fragments or variants that can be introduced into the recombinant host cells are codon-optimized with respect to the intended recipient recombinant host cell. As used herein the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0.

The heterologous nucleic acid molecule can be introduced in the host cell using a vector. A “vector,” e.g., a “plasmid”, “cosmid” or“artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The present disclosure also provides nucleic acid molecules that are hybridizable to the complement nucleic acid molecules encoding the heterologous polypeptides as well as variants or fragments. A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified, e.g., in Sambrook, J., Fritsch, E. F. and Maniatis, T. MOLECULAR CLONING: A LABORATORY MANUAL, Second Edition, Cold Spring Harbor Laboratory Press, Cold Spring Harbor (1989), particularly Chapter 11 and Table 11.1 therein. The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. For more stringent conditions, washes are performed at higher temperatures in which the washes are identical to those above except for the temperature of the final two 30 min washes in 0.2×SSC, 0.5% SDS are increased to 60° C. Another set of highly stringent conditions uses two final washes in 0.1×SSC, 0.1% SDS at 65° C. An additional set of highly stringent conditions are defined by hybridization at 0.1×SSC, 0.1% SDS, 65° C. and washed with 2×SSC, 0.1% SDS followed by 0.1×SSC, 0.1% SDS.

Hybridization requires that the two nucleic acid molecules contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived. For hybridizations with shorter nucleic acids, i.e., oligonucleotides, the position of mismatches becomes more important, and the length of the oligonucleotide determines its specificity. In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least 30 nucleotides. Furthermore, the skilled artisan will recognize that the temperature and wash solution salt concentration may be adjusted as necessary according to factors such as length of the probe.

### Combination of a First Chimeric Polypeptide Having Alpha-Amylase Activity and a Second Polypeptide Having Glucoamylase Activity

Chimeric polypeptides having the alpha-amylase activity of the present disclosure can be combined with polypeptides having glucoamylase activity to improve saccharification. In some embodiments, the chimeric polypeptides having the alpha-amylase activity and the polypeptides having glucoamylase activity are used in the process for hydrolyzing starch. The process for hydrolyzing starch involves hydrolyzing starch from a medium comprising starch, such as raw starch. For example, the medium is derived from corn or sugar cane, or derivatives thereof.

In an embodiment, the hydrolysis of starch is for the production of a fermentation product, such as ethanol. The balance between hydrolysis and fermentation keeps the presence of reducing sugars low and reduces the osmotic stress on the recombinant host cell. In addition to increasing process efficiency, recombinant expression of these distinct but complimentary enzymes is able to reduce the need for addition of expensive amylase mixtures, as well as reduce the need for the energy-intensive step of heating the raw material to temperatures approaching 180° C. (e.g., gelatinization) prior to fermentation.

The polypeptides having glucoamylase activity can be provided in a (substantially) purified form. As used in the context of the present disclosure, the expression “purified form” refer to the fact that the polypeptides have been physically dissociated from at least one components required for their production (a host cell or a host cell fragment). A purified form of the polypeptide of the present disclosure can be a cellular extract of a host cell expressing the polypeptide being enriched for the polypeptide of interest (either by positive or negative selection). The expression “substantially purified form” refer to the fact that the polypeptides have been physically dissociated from the majority of components required for their production. In an embodiment, a polypeptide in a substantially purified form is at least 90%, 95%, 96%, 97%, 98% or 99% pure. Alternatively or in combination, the polypeptides having glucoamylase activity can be provided by a recombinant host cell capable of expressing, in a recombinant fashion, the polypeptides.

In an embodiment, the chimeric polypeptides having alpha-amylase activity are used in a substantially purified form in combination with the polypeptides having glucoamylase activity. In such embodiment, the substantially purified chimeric polypeptides having alpha-amylase activity can be used to supplement a fermentation medium comprising starch and a microorganism capable of fermenting glucose into ethanol (“fermentation microorganism”). Still in such embodiment, the source of the chimeric polypeptides having alpha-amylase activity can be provided exclusively from the substantially purified chimeric polypeptides having alpha-amylase activity, or in combination with a recombinant host cell, to be included in the fermentation medium, expressing the chimeric polypeptides having alpha-amylase activity in a recombinant fashion. The polypeptides having glucoamylase activity can be provided, in the fermentation medium, in a substantially purified form and/or expressed from the recombinant host cell in a recombinant fashion. The recombinant host cell (expressing the chimeric polypeptides having alpha-amylase activity and/or the polypeptides having glucoamylase activity) can be the fermentation microorganism. In still a further embodiment, when the chimeric polypeptides having alpha-amylase activity are provided, in the fermentation medium, in a substantially purified form, the polypeptides having glucoamylase activity are expressed, in the fermentation medium, from a recombinant host cell in a recombinant fashion. In yet another embodiment, the only enzymatic supplementation that is used when the polypeptides having glucoamylase activity are expressed from a recombinant host is the chimeric polypeptide having alpha-amylase activity as described herein (e.g., no additional exogenous amylolytic enzymes are added to the fermentation medium).

In an embodiment, the chimeric polypeptides having alpha-amylase activity can be expressed from a recombinant host cell in a recombinant fashion in combination with the polypeptides having glucoamylase activity. In such embodiment, the recombinant host cell expressing the chimeric polypeptides having alpha-amylase activity are added to a fermentation medium comprising starch. If the recombinant host expressing the chimeric polypeptides having alpha-amylase activity is capable of fermenting glucose into ethanol, then no additional fermentation microorganism is required (but can nevertheless be added). However, if the recombinant host expressing the chimeric polypeptides having alpha-amylase activity is not capable of fermentation glucose into ethanol, then it is necessary to include a fermentation organism capable of fermenting glucose into ethanol in the fermentation medium. Still in such embodiment, in the fermentation medium, the source of the chimeric polypeptides having alpha-amylase activity can be provided exclusively from recombinant host cell expressing the chimeric polypeptides having alpha-amylase activity in a recombinant fashion or in combination with the substantially purified chimeric polypeptides having alpha-amylase activity. In this embodiment, the polypeptides having glucoamylase activity can be provided, in the fermentation medium, in a substantially purified form and/or expressed from a recombinant host cell in a recombinant fashion. The recombination host cell (expressing the chimeric polypeptides having alpha-amylase activity and/or the polypeptides having glucoamylase activity) can be the fermentation microorganism. In still a further embodiment, when the chimeric polypeptides having alpha-amylase activity are expressed, in the fermentation medium, from a recombinant host cell in a recombinant fashion, the polypeptides having glucoamylase activity are expressed, in the fermentation medium, from the same or a different recombinant host cell in a recombinant fashion. In yet another embodiment, when both the chimeric polypeptides having alpha-amylase activity and the polypeptides having glucoamylase activity are expressed from a recombinant source (the same or different) no additional exogenous amylolytic enzyme is included in the fermentation medium during the fermentation.

As indicated herein the recombinant host cells described herein can include additional modifications that those necessary to allow the expression of the chimeric polypeptides having alpha-amylase activity and/or the polypeptides having glucoamylase activity.

The present application also provides a population of recombinant host cells expressing the chimeric polypeptides having alpha-amylase activity to be combined with polypeptides having glucoamylase activity. In an embodiment, the population of host cells is homogeneous, i.e., each recombinant host cell of the population comprises the same genetic modifications allowing for the expression of the chimeric polypeptides having alpha-amylase activity. For example, the homogeneous population of cells can comprise recombinant host cells expressing the chimeric polypeptides having alpha-amylase activity and can optionally further express the polypeptides having glucoamylase activity. In yet another example, the homogenous population of cells can comprise recombinant host cells expressing the chimeric polypeptides having alpha-amylase activity in combination with polypeptides having glucoamylase activity in a substantially purified form.

In another embodiment, the population of host cells is heterogeneous, i.e., the population comprises two or more subpopulations of recombinant host cells wherein each members of the same subpopulation of recombinant host cells comprises at least one common genetic modification(s) which differ from the at least other common genetic modification(s) shared amongst the other subpopulation of recombinant cells. For example, in the heterogeneous population of recombinant cells, the first subpopulation of recombinant cells can include a genetic modification allowing for the expression of the chimeric polypeptides having alpha-amylase activity but not for the polypeptides having glucoamylase activity while the second subpopulations of recombinant cells include a genetic modification allowing for the expression of the polypeptides having glucoamylase activity but not for the chimeric polypeptides having alpha-amylase activity. In such embodiment, the second subpopulation of cells can include additional genetic modification, for example, a genetic modification for reducing the production of one or more native enzymes that function to produce glycerol or regulate glycerol synthesis and/or a genetic modification for reducing the production of one or more native enzymes that function to catabolize formate.

In the embodiment in which the heterogeneous population comprises a first subpopulation expressing the chimeric polypeptides having alpha-amylase activity and a second subpopulation expressing the polypeptides having glucoamylase activity. In such embodiment, at the start of the fermentation, the ratio of the secreted chimeric alpha-amylase to glucoamylase, in a fermentation medium which has not been supplemented with a purified enzymatic preparation, is about 1:2, 1:3, 1:4, 1:5, 1:6, 1:7, 1:8, 1:9, 1:10, 1:11, 1:12, 1:13, 1:14, 1:15, 1:16, 1:17, 1:18, 1:19 or 1:20.

### Yeast Products and Processes for Making Yeast Products

The recombinant yeast cells of the present disclosure can be used in the preparation of a yeast product which can ultimately be used as an additive to improve the yield of a fermentation by a fermenting yeast cell. In some embodiments, the yeast products made by the process of the present disclosure can comprise at least 0.1% (in dry weight percentage) of the heterologous enzyme when compared the total proteins of the yeast product. The yeast products of the present disclosure can include one or more heterologous enzymes as described herein. In another embodiment, the present disclosure provides processes as well as yeast products having a specific minimal enzymatic activity and/or a specific range of enzymatic activity. Advantageously, the chimeric polypeptides present in the yeast products can be concentrated during processing and can remain biologically active to perform its intended function in the yeast products.

When the yeast product is an inactivated yeast product, the process for making the yeast product broadly comprises two steps: a first step of providing propagated recombinant yeast host cells and a second step of lysing the propagated yeast host cells for making the yeast product. The process for making the yeast product can include an optional separating step and an optional drying step. In some embodiments, the process can include providing the propagated recombinant yeast host cells which have been propagated on molasses. Alternatively, the process can include providing the propagated recombinant yeast host cells are propagated on a medium comprising a yeast extract. In some embodiment, the process can further comprises propagating the recombinant yeast host cells (on a molasses or YPD medium for example).

In some embodiments, the propagated recombinant yeast host cells can be lysed using autolysis (which can be optionally be performed in the presence of additional exogenous enzymes) or homogenized (for example using a bead milling, bead beating or a high pressure homogenizing technique).

In some embodiments, the propagated recombinant yeast host cells can be lysed using autolysis. For example, the propagated recombinant yeast host cells may be subject to a combined heat and pH treatment for a specific amount of time (e.g., 24 h) in order to cause the autolysis of the propagated recombinant yeast host cells to provide the lysed recombinant yeast host cells. For example, the propagated recombinant cells can be submitted to a temperature of between about 40° C. to about 70° C. or between about 50° C. to about 60° C. The propagated recombinant cells can be submitted to a temperature of at least about 40° C., 41° C. 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C. or 70° C. Alternatively or in combination the propagated recombinant cells can be submitted to a temperature of no more than about 70° C., 69° C., 68° C., 67° C., 66° C., 65° C., 64° C., 63° C., 62° C., 61° C., 60° C., 59° C., 58° C., 57° C., 56° C., 55° C., 54° C., 53° C., 52° C., 51° C., 50° C., 49° C., 48° C., 47° C., 46° C., 45° C., 44° C., 43° C., 42° C., 41° C. or 40° C. In another example, the propagated recombinant cells can be submitted to a pH between about 4.0 and 8.5 or between about 5.0 and 7.5. The propagated recombinant cells can be submitted to a pH of at least about, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4 or 8.5. Alternatively or in combination, the propagated recombinant cells can be submitted to a pH of no more than 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3., 5.2, 5.1, 5.0, 4.9, 4.8, 4.7, 4.6 or 4.5.

In some embodiments, the recombinant yeast host cells can be homogenized (for example using a bead-milling technique, a bead-beating or a high pressure homogenization technique) and as such the process for making the yeast product comprises an homogenizing step.

The process can also include a drying step. The drying step can include, for example, with spray-drying and/or fluid-bed drying. When the yeast product is an autolysate, the process may include directly drying the lysed recombinant yeast host cells after the lysis step without performing an additional separation of the lysed mixture.

To provide additional yeast products, it may be necessary to further separate the components of the lysed recombinant yeast host cells. For example, the cellular wall components (referred to as a “Insoluble fraction”) of the lysed recombinant yeast host cell may be separated from the other components (referred to as a “soluble fraction”) of the lysed recombinant yeast host cells. This separating step can be done, for example, by using centrifugation and/or filtration. The process of the present disclosure can include one or more washing step(s) to provide the cell walls or the yeast extract. The yeast extract can be made by drying the soluble fraction obtained.

In an embodiment of the process, the soluble fraction can be further separated prior to drying. For example, the components of the soluble fraction having a molecular weight of more than 10 kDa can be separated out of the soluble fraction. This separation can be achieved, for example, by using filtration (and more specifically ultrafiltration). When filtration is used to separate the components, it is possible to filter out (e.g., remove) the components having a molecular weight less than about 10 kDa and retain the components having a molecular weight of more than about 10 kDa. The components of the soluble fraction having a molecular weight of more than 10 kDa can then optionally be dried to provide a retentate as the yeast product.

When the yeast product is an active/semi-active product, it can be submitting to a concentrating step, e.g. a step of removing part of the propagation medium from the propagated yeast host cells. The concentrating step can include resuspending the concentrated and propagated yeast host cells in the propagation medium (e.g., unwashed preparation) or a fresh medium or water (e.g., washed preparation).

In the process described herein, the yeast product is provided as an inactive form or is created during the liquefaction/fermentation process. The yeast product can be provided in a liquid, semi-liquid or dry form. In some embodiments, the inactivated yeast product is provided in the form of a cream yeast. As used herein, “cream yeast” refers to an active or semi-active yeast product obtained following the propagation of the yeast host cells.

In an aspect, the chimeric polypeptides having alpha-amylase activity and recombinant yeast host cells may be provided in a composition that additionally includes starch and/or a glucoamylase. In some embodiments, the composition can be provided in a liquefaction medium, a liquefied medium or a fermentation medium. A liquefaction medium comprises relatively intact starch molecules. A liquefied medium is a medium obtained after a liquefaction step in which the starch has been optionally heated and at least part of the starch molecules have been hydrolyzed. The viscosity of the liquefied medium is lower than the viscosity of the liquefaction medium prior to the liquefaction step. A fermentation medium comprises a liquefied medium to which a fermenting organism (such as a fermenting yeast cell) capable of metabolizing starch to produce a fermentation product (e.g., ethanol and CO₂) has been added. During the fermentation step, the starch molecules of the fermentation medium can be further hydrolyzed.

The process of the present disclosure also include a process for isolating the chimeric polypeptides having alpha-amylase activity from the recombinant host cell. The polypeptides obtained from such process can be used during the liquefaction step and thus introduced in the liquefaction medium and/or fermentation medium. The process includes removing at least some (and in an embodiment, the majority) of the components of the recombinant yeast host cell from the heterologous polypeptides having alpha-amylase activity. Alternatively or in combination, the process includes selecting the chimeric polypeptides having alpha-amylase activity from the components of the recombinant host cells. The process can include a centrifugation step, a filtration step, a washing step and/or a drying step to provide the chimeric polypeptides having alpha-amylase activity in a purified form. In embodiments in which the heterologous polypeptides having alpha-amylase activity are expressed intracellularly or associated with the recombinant host cell's membrane, the process can include lysing the recombinant host cells. In embodiments in which the heterologous polypeptides having alpha-amylase activity are expressed associated with the recombinant yeast host cell's membrane, the process can include disrupting the recombinant host cells' membranes to purify the heterologous polypeptides having alpha-amylase activity.

### Process for Hydrolyzing Starch

The chimeric polypeptides and recombinant host cells described herein can be used to hydrolyze (e.g., saccharify) starch and/or dextrins into smaller molecules (such as glucose). In an embodiment, the polypeptides and recombinant host cells described herein can be used to hydrolyze start for making fermentation product, such as ethanol. The polypeptides and recombinant host cells described herein hydrolyze starch into glucose to allow a concomitant or subsequent fermentation of glucose into ethanol. The polypeptides can be used in a substantially purified form as an additive to a fermentation process. Alternatively or in combination, the polypeptides can be expressed from one or more recombinant host cell during the fermentation process.

The process comprises combining a substrate to be hydrolyzed (optionally included in a liquefaction medium) with the recombinant host yeast cells expressing the polypeptides, a yeast product obtained from the recombinant yeast host cell and/or with the polypeptides in a substantially purified form. At this stage, further purified enzymes, such as, for example, non-thermostable alpha-amylases can be added also be included in the liquefaction medium.

The biomass that can be fermented with the recombinant host cell described herein includes any type of biomass known in the art and described herein. For example, the biomass can include, but is not limited to, starch, sugar and lignocellulosic materials. Starch materials can include, but are not limited to, mashes such as corn, wheat, rye, barley, rice, or milo. Sugar materials can include, but are not limited to, sugar beets, artichoke tubers, sweet sorghum, molasses or cane. The terms “lignocellulosic material”, “lignocellulosic substrate” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants and sugar-processing residues. The terms “hemicellulosics”, “hemicellulosic portions” and “hemicellulosic fractions” mean the non-lignin, non-cellulose elements of lignocellulosic material, such as but not limited to hemicellulose (i.e., comprising xyloglucan, xylan, glucuronoxylan, arabinoxylan, mannan, glucomannan and galactoglucomannan), pectins (e.g., homogalacturonans, rhamnogalacturonan I and II, and xylogalacturonan) and proteoglycans (e.g., arabinogalactan-protein, extensin, and pro line-rich proteins).

In a non-limiting example, the lignocellulosic material can include, but is not limited to, woody biomass, such as recycled wood pulp fiber, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; succulents, such as but not limited to, agave; and forestry wastes, such as but not limited to, recycled wood pulp fiber, sawdust, hardwood (e.g., poplar, oak, maple, birch, willow), softwood, or any combination thereof. Lignocellulosic material may comprise one species of fiber; alternatively, lignocellulosic material may comprise a mixture of fibers that originate from different lignocellulosic materials. Other lignocellulosic materials are agricultural wastes, such as cereal straws, including wheat straw, barley straw, canola straw and oat straw; corn fiber: stovers, such as corn stover and soybean stover; grasses, such as switch grass, reed canary grass, cord grass, and miscanthus; or combinations thereof.

Substrates for cellulose activity assays can be divided into two categories, soluble and insoluble, based on their solubility in water. Soluble substrates include cellodextrins or derivatives, carboxymethyl cellulose (CMC), or hydroxyethyl cellulose (HEC). Insoluble substrates include crystalline cellulose, microcrystalline cellulose (Avicel), amorphous cellulose, such as phosphoric acid swollen cellulose (PASC), dyed or fluorescent cellulose, and pretreated lignocellulosic biomass. These substrates are generally highly ordered cellulosic material and thus only sparingly soluble.

It will be appreciated that suitable lignocellulosic material may be any feedstock that contains soluble and/or insoluble cellulose, where the insoluble cellulose may be in a crystalline or non-crystalline form. In various embodiments, the lignocellulosic biomass comprises, for example, wood, corn, corn stover, sawdust, bark, molasses, sugarcane, leaves, agricultural and forestry residues, grasses such as switchgrass, ruminant digestion products, municipal wastes, paper mill effluent, newspaper, cardboard or combinations thereof.

Paper sludge is also a viable feedstock for lactate or acetate production. Paper sludge is solid residue arising from pulping and paper-making, and is typically removed from process wastewater in a primary clarifier. The cost of disposing of wet sludge is a significant incentive to convert the material for other uses, such as conversion to ethanol. Processes provided by the present invention are widely applicable. Moreover, the sacchariflcation and/or fermentation products may be used to produce ethanol or higher value added chemicals, such as organic acids, aromatics, esters, acetone and polymer intermediates.

The process comprises combining a substrate to be hydrolyzed (optionally included in a fermentation medium) with the recombinant host cells expressing the polypeptides and/or with the polypeptides in a substantially purified form. In an embodiment, the substrate to be hydrolyzed is a lignocellulosic biomass and, in some embodiments, it comprises starch (in a gelatinized or raw form). In some embodiments, the substrate is raw starch. In some embodiments, the raw starch is derived from corn or sugar cane, or a derivative therefrom. In some embodiments, the use of recombinant host cells or the purified polypeptides limits or avoids the need of adding additional external source of purified enzymes during fermentation to allow the breakdown of starch. The expression of the polypeptides in a recombinant host cell is advantageous because it can reduce or eliminate the need to supplement the fermentation medium with external source of purified enzymes (e.g., glucoamylase and/or chimeric alpha-amylase) while allowing the fermentation of the lignocellulosic biomass into a fermentation product (such as ethanol).

The chimeric polypeptides, recombinant host cells expressing same and composition comprising same described herein can be used to increase the production of a fermentation product during fermentation. The chimeric polypeptides, recombinant host cells or compositions of the present disclosure can be used prior to, during and/or after the heating step to gelatinize the starch. The process comprises combining a substrate to be hydrolyzed (optionally included in a fermentation medium) with the chimeric polypeptide (either in a purified form, in a composition or expressed in a recombinant host cell). In an embodiment, the substrate to be hydrolyzed is a lignocellulosic biomass. In some embodiments, the substrate comprises starch (in a gelatinized or raw form). In still another embodiment, the substrate comprises raw starch and the process includes heating (gelatinizing) the starch prior to and/or during a propagation phase of fermentation.

In some embodiments, the liquefaction of starch occurs in the presence of chimeric polypeptide, the recombinant host cells or the compositions. In some embodiments, the liquefaction of starch is maintained at a temperature of between about 25° C. and about 60° C. for a period of time to allow for proper gelatinization and hydrolysis of the crystalline starch. In an embodiment, the liquefaction occurs at a temperature of at least about 25° C., 30° C., 35° C., 40° C., 45° C., 50° C. or 55° C. Alternatively or in combination, the liquefaction occurs at a temperate of no more than about 60° C., 55° C., 50° C., 45° C., 40° C., 35° C., 30° C. or 25° C.

The chimeric polypeptides having alpha-amylase activity described herein can be used to increase the production of a fermentation product during fermentation. The process comprises combining a substrate to be hydrolyzed (optionally included in a fermentation medium) with the chimeric polypeptide having alpha-amylase activity (either in a purified form or expressed in a recombinant host cell) and the polypeptide having glucoamylase activity (either in a purified form or expression in a recombinant host cell). In an embodiment, the process can comprise combining the substrate with an heterologous population of recombinant host cells as described herein. In an embodiment, the substrate to be hydrolyzed is a lignocellulosic biomass and, in some embodiments, it comprises starch (in a gelatinized or raw form). In still another embodiment, the substrate comprises raw starch (such as raw starch derived from corn) and the process excludes the step of heating (gelatinizing) the starch prior to fermentation and/or the step of adding other enzymes, such as other alpha-amylases, than those described herein. This embodiment is advantageous because it can reduce or eliminate the need to supplement the fermentation medium with external source of purified enzymes (e.g., glucoamylase and/or alpha-amylase) while allowing the fermentation of the lignocellulosic biomass into a fermentation product (such as ethanol). However, in some circumstances, it may be advisable to supplement the medium with a chimeric polypeptide having alpha-amylase activity in a purified form. Such polypeptide can be produced in a recombinant fashion in a recombinant host cell.

The production of ethanol can be performed during a fermentation with a fermenting organism at temperatures of at least about 25° C., about 28° C., about 30° C., about 31° C., about 32° C., about 33° C., about 34° C., about 35° C., about 36° C., about 37° C., about 38° C., about 39° C., about 40° C., about 41° C., about 42° C., or about 50° C. In some embodiments, when a thermotolerant yeast cell is used in the process, the process can be conducted at temperatures above about 30° C., about 31° C., about 32° C., about 33° C., about 34° C., about 35° C., about 36° C., about 37° C., about 38° C., about 39° C., about 40° C., about 41° C., about 42° C., or about 50° C.

In some embodiments, the process can be used to produce ethanol at a particular rate. For example, in some embodiments, ethanol is produced at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, or at least about 500 mg per hour per liter.

Ethanol production can be measured using any method known in the art. For example, the quantity of ethanol in fermentation samples can be assessed using HPLC analysis. Many ethanol assay kits are commercially available that use, for example, alcohol oxidase enzyme based assays.

The process of the present disclosure also include a process for isolating the polypeptides having alpha-amylase activity from the recombinant yeast host cell. The polypeptides obtained from such process can be used during the liquefaction step and thus introduced in the liquefaction medium. The process includes removing at least some (and in an embodiment, the majority) of the components of the recombinant yeast host cell from the heterologous polypeptides having alpha-amylase activity. Alternatively or in combination, the process includes selecting the heterologous polypeptides having alpha-amylase activity from the components of the recombinant yeast host cells. The process can include a centrifugation step, a filtration step, a washing step and/or a drying step to provide the heterologous polypeptides having alpha-amylase activity in a purified form. In embodiments in which the heterologous polypeptides having alpha-amylase activity are expressed intracellularly or associated with the recombinant yeast host cell's membrane, the process can include lysing the recombinant yeast host cells. In embodiments in which the heterologous polypeptides having alpha-amylase activity are expressed associated with the recombinant yeast host cell's membrane, the process can include disrupting the recombinant yeast host cells' membranes to purify the heterologous polypeptides having alpha-amylase activity.

The present invention will be more readily understood by referring to the following examples which are given to illustrate the invention rather than to limit its scope.

### Example—Chimeric Alpha-Amylase Strains

This example describes a process for engineering a chimeric alpha-amylase enzyme by fusing the alpha amylase to a starch binding domain to improve its activity on raw starch substrates. When simultaneously secreted with a glucoamylase, this chimeric alpha-amylase allows for significant reductions in exogenous enzyme inputs.

Strains Saccharomyces cerevisiae were constructed (see table 2) to express an heterologous gene coding for an alpha-amylase (see table 1), more specifically, a mutant version of SE85 was developed by attaching a linker and a starch binding domain (SBD) region of the Aspergillus niger glucoamylase G1 to the the C-terminus of SE85 alpha-amylase from Bacillus amyloliquefaciens.

TABLE 1
Description of relevant enzymes.

| Enzyme | Description |
|---|---|
| SE85 | Bacillus amyloliquefaciens amyE alpha-amylase |
| | (SEQ ID NO: 1 and 2) |
| MP1032 | Chimeric protein comprised of SE85 and the linker and |
| | SBD regions from Aspergillus niger G1 glucoamylase |
| | (SEQ ID NO: 4 and 5) |

TABLE 2
Description of relevant strains.

| Strain | Description |
|---|---|
| M9900 | Strain expression 2 copies of SE85 alpha-amylase |
| M15747 | Strain expressing 2 copies of chimeric alpha-amylase/ |
| | SBD MP1032 |

Cell growth. Cells were grown overnight in 5 mL YPD (10 g/L yeast extract, 20 g/L bacteriological peptone, 40 g/L glucose). One (1) mL of whole culture as harvested and cells were pelleted by centrifugation. Cell-free supernatant was removed and saved for later analysis.

Alpha-amylase assay. Alpha-amylase activity was measured by adding 150 μL cell-free supernatant to 150 μL 4% (w/v) corn flour in 50 mM sodium acetate pH 5. The reaction was incubated at 35° C. for 2 hours, at which time 50 μL was sampled and measured for reducing sugars via the 3,5 dinitrosalicylic acid (DNS) method.

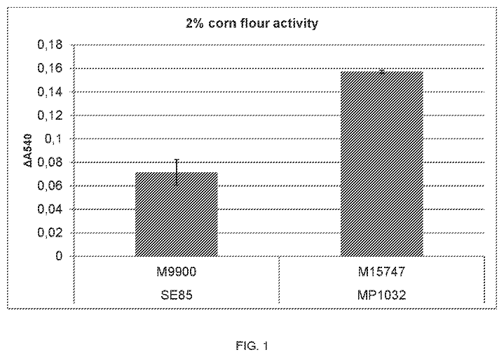

This chimeric protein is identified as MP1032, when expressed from S. cerevisiae yeast strain M15747 exhibited a two-fold improvement in secreted activity on corn flour than alpha-amylase SE85 expressed from S. cerevisiae strain M9900 (FIG. 1).

### REFERENCES

Ghang, Dong-Myeong, et al. “Efficient one-step starch utilization by industrial strains of Saccharomyces cerevisiae expressing the glucoamylase and α-amylase genes from Debaryomyces occidentalis.” Biotechnology letters 29.8 (2007): 1203-1208.
Birol, Gülnur, et al. “Ethanol production and fermentation characteristics of recombinant Saccharomyces cerevisiae strains grown on starch.” Enzyme and microbial technology 22.8 (1998): 672-677.
Shigechi, Hisayori, et al. “Direct production of ethanol from raw corn starch via fermentation by use of a novel surface-engineered yeast strain codisplaying glucoamylase and α-amylase.” Applied and Environmental Microbiology 70.8 (2004): 5037-5040.
Juge, Nathalie, et al. “The activity of barley α-amylase on starch granules is enhanced by fusion of a starch binding domain from Aspergillus niger glucoamylase.” Biochimica et Biophysica Acta (BBA)-Proteins and Proteomics 1764.2 (2006): 275-284.
Ohdan, Kohji, et al. “Introduction of Raw Starch-Binding Domains into Bacillus subtilis α-Amylase by Fusion with the Starch-Binding Domain of Bacillus Cyclomaltodextrin Glucanotransferase.” Applied and environmental microbiology 66.7 (2000): 3058-3064.
Catlett, Michael G. Yeast strains suitable for saccharification and fermentation expressing glucoamylase and/or alpha-amylase.
Patent Application PCT/US2016/061887 published under WO/2017/087330

What is claimed is:

1. A chimeric polypeptide having alpha-amylase activity, the chimeric polypeptide comprising a polypeptide having alpha-amylase activity (AA) associated with a starch binding domain (SBD) moiety.

2. The chimeric polypeptide of claim 1, wherein the polypeptide having AA is from a Bacillus sp. alpha-amylase, a variant thereof, or a fragment thereof.

3. The chimeric polypeptide of claim 2, wherein the polypeptide having AA is from a Bacillus amyloliquefaciens alpha-amylase, a variant thereof, or a fragment thereof.

4. The chimeric polypeptide of claim 3, wherein the polypeptide having AA is from a Bacillus amyloliquefaciens amyE alpha-amylase, a variant thereof, or a fragment thereof.

5. The chimeric polypeptide of claim 4, wherein the polypeptide having AA has the amino acid sequence of SEQ ID NO: 3 or 12, is a variant of the amino acid sequence of SEQ ID NO: 3 or 12, or is a fragment of the amino acid sequence of SEQ ID NO: 3 or 12.

6. The chimeric polypeptide of any one of claims 1 to 5, wherein the SBD moiety has high binding affinity to raw starch.

7. The chimeric polypeptide of any one of claims 1 to 6, wherein the SBD moiety enhances the activity of the polypeptide having AA on raw starch when compared to the activity of a polypeptide having AA and lacking the SBD moiety, the variant thereof or the fragment thereof.

8. The chimeric polypeptide of any one of claims 1 to 7, wherein the SBD moiety is derived from a glucoamylase enzyme, a variant thereof, or a fragment thereof.

9. The chimeric polypeptide of claim 8, wherein the SBD moiety is derived from an Aspergillus sp. glucoamylase, a variant thereof, or a fragment thereof.

10. The chimeric polypeptide of claim 9, wherein the SBD moiety is derived from an Aspergillus niger glucoamylase G1, a variant thereof, or a fragment thereof.

11. The chimeric polypeptide of claim 10, wherein the SBD moiety has the amino acid sequence of SEQ ID NO: 7, is a variant of the amino acid sequence of SEQ ID NO: 7, or is a fragment of the amino acid sequence of SEQ ID NO: 7.

12. The chimeric polypeptide of any one of claims 1 to 11, wherein the chimeric polypeptide comprises the amino acid sequence of SEQ ID NO: 8, is a variant of the amino acid sequence of SEQ ID NO: 8, or is a fragment of the amino acid sequence of SEQ ID NO: 8.

13. The chimeric polypeptide of any one of claims 1 to 12, further comprising a signal sequence (SS) attached to the amino terminus of the chimeric polypeptide.

14. The chimeric polypeptide of claim 13, wherein the SS has the amino acid sequence of SEQ ID NO: 6, 13, 14, or 15; is a variant of the amino acid sequence of SEQ ID NO: 6, 13, 14, or 15; or is a fragment of the amino acid sequence of SEQ ID NO: 6, 13, 14, or 15.

15. The chimeric polypeptide of claim 13, wherein the chimeric polypeptide comprises the amino acid sequence of SEQ ID NO: 5, is a variant of the amino acid sequence of SEQ ID NO: 5, or is a fragment of the amino acid sequence of SEQ ID NO: 5.

16. The chimeric polypeptide of any one of claims 1 to 15, further comprising an amino acid linker linking the AA moiety and the SBD moiety.

17. The chimeric polypeptide of claim 16, wherein the amino acid linker comprises one or more glycine residues and/or serine residues.

18. The chimeric polypeptide of claim 16, wherein the amino acid linker has the amino acid sequence of SEQ ID NO: 16, 17, 18, 19, 20, 21, 22, 23, or 24; is a variant of the amino acid sequence of SEQ ID NO: 16, 17, 18, 19, 20, 21, 22, 23, or 24; or is a fragment of the amino acid sequence of SEQ ID NO: 16, 17, 18, 19, 20, 21, 22, 23, or 24.

19. The chimeric polypeptide of any one of claims 1 to 18 being provided in a purified form or expressed from an heterologous nucleic acid molecule encoding the chimeric polypeptide in a recombinant host cell.

20. An isolated nucleic acid molecule encoding the chimeric polypeptide of any one of claims 1 to 19.

21. The isolated nucleic acid molecule of claim 20 comprising the nucleotide sequence of SEQ ID NO: 1 or 4, being a variant thereof or a fragment thereof.

22. A recombinant host cell having an heterologous nucleic acid molecule encoding the chimeric polypeptide of any one of claims 1 to 19 or defined in claim 20 or 21.

23. The recombinant host cell of claim 22 being from the genus Saccharomyces sp.

24. The recombinant host cell of claim 23 being from the species Saccharomyces cerevisiae.

25. A purified, isolated and/or recombinant chimeric polypeptide obtained from a recombinant host cell of any one of claims 22 to 24.

26. A composition comprising the recombinant host cell of any one of claims 22 to 24 or the purified, isolated and/or recombinant chimeric polypeptide of claim 25 and at least one of a glucoamylase or starch.

27. A yeast product made from the recombinant yeast host cell of any one of claims 22 to 24, comprising the purified, isolated and/or recombinant chimeric polypeptide of claim 25 or the composition of claim 26.

28. The yeast product of claim 27 being an inactivated yeast product.

29. The yeast product of claim 28 being a yeast extract.

30. A process for hydrolyzing starch, the process comprising contacting the chimeric polypeptide of any one of claims 1 to 19, the recombinant host cell of any one of claims 22 to 24, the purified, isolated and/or recombinant chimeric polypeptide of claim 25, the composition of claim 26 or the yeast product of any one of claims 27 to 29 with a medium comprising starch.

31. The process of claim 30, wherein the medium comprises raw starch.

32. The process of claim 30 or 31, wherein the medium is derived from corn.

33. The process of any one of claims 30 to 32, comprising adding the recombinant host cell, the purified, isolated and/or recombinant polypeptide, the composition or the yeast product to a liquefaction medium.

34. The process of claim 33 comprising maintaining the liquefaction medium at a temperature of between about 25° C. and 60° C. during a period of time to obtain a liquefied medium.

35. The process of claim 34 for making a fermentation product from the liquefied medium.

36. The process of claim 35, further comprising fermenting the liquefied medium with a fermenting yeast cell to obtain the fermented product.

37. The process of claim 36, wherein the fermentation product is ethanol.


SEQUENCE LISTING (24 sequences)

SEQ ID NO: 1 - DNA, 1956 bp, Bacillus amyloliquefaciens
atgttgttgcaagccttcttgtttttgttggctggttttgctgctaagatttctgctggtccagctgctgctaatgctgaaactgctaacaaatctaacaaggttactgcctcctctgttaagaatggtactattttacatgcctggaactggtctttcaacactttgactcaaaacatgaaggacattagagatgctggttacgctgctattcaaacctctccaatcaatcaagtcaaagaaggtaatcaaggtgacaagtctatgagaaattggtactggttgtaccaacctacctcttaccaaatcggtaatagatacttgggtactgaacaagaattcaaggatatgtgtgctgctgctgaaaagtatggtgttaaggttatagttgacgccgttattaaccataccacatctgattatggtgccatctctgacgaaatcaagagaattccaaattggactcatggtaacacccaaatcaagaattggtctgatagatgggatgtcacccaaaattctttgttgggtttgtacgattggaatacccaaaacaccgaagttcaagtctacttgaagagattcttggaaagagctttgaacgatggtgctgatggttttagatatgatgccgccaaacatatcgaattgccagatgatggtaattacggttctcaattctggccaaatattaccaacacttccgctgaatttcaatacggtgaaatattgcaagactccgcttctagagatactgcttatgctaattacatgaacgttaccgcttctaactacggtcattctattagatctgccttgaagaacagaaacttgtccgtttccaacatttctcattacgcctctgatgtttctgccgataagttggttacttgggttgaatctcatgatacctacgctaacgatgatgaagaatctacttggatgtccgatgatgatattagattgggttgggctgttatcggttctagatctggttctactcctttgtttttctcaagacctgaaggtggtggtaatggtgttagattcccaggtaaatctcaaattggtgatagaggttctgccttgtttaaggatcaagctattactgctgttaacaccttccataatgttatggctggtcaaccagaagaattgtctaatccaaacggtaacaatcaagttttcatgaatcaaagaggttccaagggtgttgttttggctaatgcaggttcttcttccgttactattaacacctctgctaaattgcctgatggtagatacgataatagagctggtgctggttcttttcaagttgctaatggtaaattgaccggtactatcaatgctagatctgctgctgtcttgtacccagatgatattggtaatgctccacacgtctttttggaaaactatcaaactggtgccgttcactctttcaacgatcaattgactgttaccttgagagctaatgctaagactactaaggccgtctaccaaatcaacaacggtcaacaaactgctttcaaagatggtgacagattgaccattggtaagggtgatcctattggtactacctacaacattaagttgactggtactaatggtgaaggtgctgctagaactcaagaatacactttcgttaagaaggatccatcccaaactaacatcatcggttaccaaaatccagatcattggggtcaagttaacgcctacatctacaaacatgatggtggtagagctattgaattgactggttcttggccaggtaaagctatgactaagaatgctaacggtatgtacacattgaccttgccagaaaacactgatacagctaacgctaaggttatctttaacaacggttctgctcaagtcccaggtcaaaatcaaccaggttttgattacgttcaaaacggtttgtacaacaactctggtttgaacggttatttgccacac

SEQ ID NO: 2 - protein, 652 aa, Bacillus amyloliquefaciens
MLLQAFLFLLAGFAAKISAGPAAANAETANKSNKVTASSVKNGTILHAWNWSFNTLTQNMKDIRDAGYAAIQTSPINQVKEGNQGDKSMRNWYWLYQPTSYQIGNRYLGTEQEFKDMCAAAEKYGVKVIVDAVINHTTSDYGAISDEIKRIPNWTHGNTQIKNWSDRWDVTQNSLLGLYDWNTQNTEVQVYLKRFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNTFHNVMAGQPEELSNPNGNNQVFMNQRGSKGVVLANAGSSSVTINTSAKLPDGRYDNRAGAGSFQVANGKLTGTINARSAAVLYPDDIGNAPHVFLENYQTGAVHSFNDQLTVTLRANAKTTKAVYQINNGQQTAFKDGDRLTIGKGDPIGTTYNIKLTGTNGEGAARTQEYTFVKKDPSQTNIIGYQNPDHWGQVNAYIYKHDGGRAIELTGSWPGKAMTKNANGMYTLTLPENTDTANAKVIFNNGSAQVPGQNQPGFDYVQNGLYNNSGLNGYLPH

SEQ ID NO: 3 - protein, 633 aa, Bacillus amyloliquefaciens
GPAAANAETANKSNKVTASSVKNGTILHAWNWSFNTLTQNMKDIRDAGYAAIQTSPINQVKEGNQGDKSMRNWYWLYQPTSYQIGNRYLGTEQEFKDMCAAAEKYGVKVIVDAVINHTTSDYGAISDEIKRIPNWTHGNTQIKNWSDRWDVTQNSLLGLYDWNTQNTEVQVYLKRFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNTFHNVMAGQPEELSNPNGNNQVFMNQRGSKGVVLANAGSSSVTINTSAKLPDGRYDNRAGAGSFQVANGKLTGTINARSAAVLYPDDIGNAPHVFLENYQTGAVHSFNDQLTVTLRANAKTTKAVYQINNGQQTAFKDGDRLTIGKGDPIGTTYNIKLTGTNGEGAARTQEYTFVKKDPSQTNIIGYQNPDHWGQVNAYIYKHDGGRAIELTGSWPGKAMTKNANGMYTLTLPENTDTANAKVIFNNGSAQVPGQNQPGFDYVQNGLYNNSGLNGYLPH

SEQ ID NO: 4 - DNA, 2394 bp, Artificial Sequence
  Chimeric sequence
atgttgttgcaagccttcttgtttttgttggctggttttgctgctaagatttctgctggtccagctgctgctaatgctgaaactgctaacaaatctaacaaggttactgcctcctctgttaagaatggtactattttacatgcctggaactggtctttcaacactttgactcaaaacatgaaggacattagagatgctggttacgctgctattcaaacctctccaatcaatcaagtcaaagaaggtaatcaaggcgacaagtctatgagaaattggtattggttgtaccagcctacctcttaccaaatcggtaatagatatttgggtactgagcaagagttcaaggatatgtgtgctgctgctgaaaagtatggtgttaaggttatagttgacgccgttattaaccataccacatctgattatggtgccatctctgacgaaatcaagagaattccaaattggactcatggtaacacccaaatcaagaattggtctgatagatgggatgtgacccaaaattctttgttgggtctgtacgattggaatacccaaaacactgaagttcaggtctacttgaagagatttttggaaagggctttgaacgatggtgctgatggttttagatatgatgccgccaaacatatcgaattgccagatgatggtaattacggttctcaattctggcccaatattaccaatacttccgctgaatttcagtacggtgaaatcttacaagactccgcttctagagatactgcttatgctaattacatgaacgttaccgcttctaactacggtcattctattagatctgccctgaagaacagaaacttgtccgtttctaacatttcccattacgcctctgatgtttctgctgataagttggttacttgggttgaatctcatgatacctacgctaacgatgatgaagaatctacttggatgtccgatgacgatattagattaggttgggctgttatcggttctagatctggttctactcctttgtttttctcaagacctgaaggtggtggtaatggtgttagattcccaggtaaatctcaaattggtgatagaggttctgctttgtttaaggatcaagctattactgccgttaacacctttcataatgttatggctggtcagccagaagaattgtctaatccaaatggtaacaaccaggttttcatgaatcaaaggggttctaagggtgttgttttggctaatgcaggttcttcttccgttactattaacacctctgctaaattgcctgatggtagatacgataatagagctggtgctggttcttttcaagttgctaatggtaaattgaccggtactatcaatgctagatctgctgctgtcttgtacccagatgatattggtaacgctccacatgttttcttggaaaactatcaaactggtgccgttcactctttcaacgatcaattgactgttaccttgagagctaatgctaagactactaaggccgtttaccagattaacaacggtcaacaaactgctttcaaagacggtgatagattgactattggtaagggtgatccaattggtactacctacaacattaagttgactggcactaatggtgaaggtgctgctagaactcaagagtacacttttgttaagaaggatccatctcagaccaacatcatcggttatcaaaatccagatcattggggtcaagttaacgcctacatctacaaacatgatggtggtagagctattgaattgactggttcttggccaggtaaagctatgactaagaatgctaacggtatgtacacattgaccttgccagaaaacacagatacagctaacgctaaggttatcttcaacaatggttctgctcaagtcccaggtcaaaatcaacctggttttgattatgttcagaacggcttgtacaacaactctggtttgaatggttatttgccacatactggtggtactactactacagctactccaacaggttctggttctgttacttctacttctaaaactaccgctactgcttctaagacttctacctctacttcttccacttcttgtactacaccaactgctgttgctgttacttttgatttgactgctactacaacttacggcgagaacatctatttggttggttccatttctcaactaggtgattgggaaacttctgatggtattgctttgtctgcagataagtacacttcttctgatccattgtggtacgttactgttacattgccagctggtgaatcttttgagtacaagttcatcagaatcgagtccgatgattctgttgaatgggaatctgatccaaatagagagtacacagttcctcaagcttgtggtacatctactgctactgttactgatacttggaggtga

SEQ ID NO: 5 - protein, 797 aa, Artificial Sequence
  Chimeric sequence
MLLQAFLFLLAGFAAKISAGPAAANAETANKSNKVTASSVKNGTILHAWNWSFNTLTQNMKDIRDAGYAAIQTSPINQVKEGNQGDKSMRNWYWLYQPTSYQIGNRYLGTEQEFKDMCAAAEKYGVKVIVDAVINHTTSDYGAISDEIKRIPNWTHGNTQIKNWSDRWDVTQNSLLGLYDWNTQNTEVQVYLKRFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNTFHNVMAGQPEELSNPNGNNQVFMNQRGSKGVVLANAGSSSVTINTSAKLPDGRYDNRAGAGSFQVANGKLTGTINARSAAVLYPDDIGNAPHVFLENYQTGAVHSFNDQLTVTLRANAKTTKAVYQINNGQQTAFKDGDRLTIGKGDPIGTTYNIKLTGTNGEGAARTQEYTFVKKDPSQTNIIGYQNPDHWGQVNAYIYKHDGGRAIELTGSWPGKAMTKNANGMYTLTLPENTDTANAKVIFNNGSAQVPGQNQPGFDYVQNGLYNNSGLNGYLPHTGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 6 - protein, 19 aa, Artificial Sequence
  Signal sequence
MLLQAFLFLLAGFAAKISA

SEQ ID NO: 7 - protein, 108 aa, Aspergillus niger [sequence omitted]

SEQ ID NO: 8 - protein, 778 aa, Artificial Sequence
  Chimeric sequence
GPAAANAETANKSNKVTASSVKNGTILHAWNWSFNTLTQNMKDIRDAGYAAIQTSPINQVKEGNQGDKSMRNWYWLYQPTSYQIGNRYLGTEQEFKDMCAAAEKYGVKVIVDAVINHTTSDYGAISDEIKRIPNWTHGNTQIKNWSDRWDVTQNSLLGLYDWNTQNTEVQVYLKRFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNTFHNVMAGQPEELSNPNGNNQVFMNQRGSKGVVLANAGSSSVTINTSAKLPDGRYDNRAGAGSFQVANGKLTGTINARSAAVLYPDDIGNAPHVFLENYQTGAVHSFNDQLTVTLRANAKTTKAVYQINNGQQTAFKDGDRLTIGKGDPIGTTYNIKLTGTNGEGAARTQEYTFVKKDPSQTNIIGYQNPDHWGQVNAYIYKHDGGRAIELTGSWPGKAMTKNANGMYTLTLPENTDTANAKVIFNNGSAQVPGQNQPGFDYVQNGLYNNSGLNGYLPHTGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 9 - protein, 515 aa, Saccharomycopsis fibuligera
MIRLTVFLTAVFAAVASCVPVELDKRNTGHFQAYSGYTVARSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 10 - protein, 515 aa, Artificial Sequence
  A40N variant of S. fibuligera glucosamylase
MIRLTVFLTAVFAAVASCVPVELDKRNTGHFQAYSGYTVNRSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 11 - protein, 519 aa, Saccharomycopsis fibuligera
MRFGVLISVFAAIVSALPLQEGPLNKRAYPSFEAYSNYKVDRTDLETFLDKQKEVSLYYLLQNIAYPEGQFNNGVPGTVIASPSTSNPDYYYQWTRDSAITFLTVLSELEDNNFNTTLAKAVEYYINTSYNLQRTSNPSGSFDDENHKGLGEPKFNTDGSAYTGAWGRPQNDGPALRAYAISRYLNDVNSLNEGKLVLTDSGGINFSSTEDIYKNIIKPDLEYVIGYWDSTGFDLWEENQGRHFFTSLVQQKALAYAVDIAKSFDDGDFANTLSSTASTLESYLSGSDGGFVNTDVNHIVENPDLLQQNSRQGLDSATYIGPLLTHDIGESSSTPFDVDNEYVLQSYYLLLEDNKDRYFVNSAYSAGAAIGRYPEDVYNGDGSSEGNPWFLATAYAAQVPYKLAYDAKSASNDITINKINYDFFNKYIVDLSTINSAYQSSDSVTIKSGSDEFNTVADNLVTFGDSFLQVILDHINDDGSLNEQLNRYTGYSTGAYSLTWSSGALLEAIRLRNKVKALA

SEQ ID NO: 12 - protein, 626 aa, Artificial Sequence
  B. amyloliquefaciens amyE alpha-amylase with modified N-terminus
ETANKSNKVTASSVKNGTILHAWNWSFNTLTQNMKDIRDAGYAAIQTSPINQVKEGNQGDKSMRNWYWLYQPTSYQIGNRYLGTEQEFKDMCAAAEKYGVKVIVDAVINHTTSDYGAISDEIKRIPNWTHGNTQIKNWSDRWDVTQNSLLGLYDWNTQNTEVQVYLKRFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNTFHNVMAGQPEELSNPNGNNQVFMNQRGSKGVVLANAGSSSVTINTSAKLPDGRYDNRAGAGSFQVANGKLTGTINARSAAVLYPDDIGNAPHVFLENYQTGAVHSFNDQLTVTLRANAKTTKAVYQINNGQQTAFKDGDRLTIGKGDPIGTTYNIKLTGTNGEGAARTQEYTFVKKDPSQTNIIGYQNPDHWGQVNAYIYKHDGGRAIELTGSWPGKAMTKNANGMYTLTLPENTDTANAKVIFNNGSAQVPGQNQPGFDYVQNGLYNNSGLNGYLPH

SEQ ID NO: 13 - protein, 19 aa, Saccharomyces cerevisiae
MLLQAFLFLLAGFAAKISA

SEQ ID NO: 14 - protein, 18 aa, Saccharomyces cerevisiae
MQLLRCFSIFSVIASVLA

SEQ ID NO: 15 - protein, 20 aa, Aspergillus terreus
MKWTFSLLLLLSVFGQATHA

SEQ ID NO: 16 - protein, 37 aa, Aspergillus niger
TGGTTTTATPTGSGSVTSTSKTTATASKTSTSTSSTS

SEQ ID NO: 17 - protein, 38 aa, Aspergillus niger
ATGGTTTTATPTGSGSVTSTSKTTATASKTSTSTSSTS

SEQ ID NO: 18 - protein, 15 aa, Artificial Sequence
  Linker
GGGGSGGGGSGGGGS

SEQ ID NO: 19 - protein, 8 aa, Artificial Sequence
  Linker
GGGGGGGG

SEQ ID NO: 20 - protein, 40 aa, Artificial Sequence
  Linker
GGGGSGGGGSGGGGSGGGGSGGGGSGGGGSGGGGSGGGGS

SEQ ID NO: 21 - protein, 12 aa, Artificial Sequence
  Linker
GSAGSAAGSGEF

SEQ ID NO: 22 - protein, 12 aa, Artificial Sequence
  Linker
EAAKEAAKEAAK

SEQ ID NO: 23 - protein, 20 aa, Artificial Sequence
  Linker
APAPAPAPAPAPAPAPAPAP

SEQ ID NO: 24 - protein, 46 aa, Artificial Sequence
  Linker
AEAAAKEAAAKEAAAKEAAAKALEAEAAAKEAAAKEAAAKEAAAKA